(19) United States
(12) Patent Application Publication
(10) Pub. No.: US 2025/0367191 A1
(43) Pub. Date: Dec. 4, 2025

(54) TREATMENT OF MITOCHONDRIAL DISEASES WITH A CNS-PENETRANT SGC STIMULATOR ZAGOCIGUAT

(71) Applicant: Tisento Therapeutics Inc., Cambridge, MA (US)

(72) Inventors: Chad Edward Glasser, Somerville, MA (US); Christopher John Winrow, Sudbury, MA (US)

(21) Appl. No.: 18/872,750

(22) PCT Filed: Jun. 9, 2023
(86) PCT No.: PCT/US2023/024932

§ 371 Date: Dec. 6, 2024

Related U.S. Application Data
(60) Provisional application No. 63/350,708, filed on Jun. 9, 2022.
(60) Provisional application No. 63/410,829, filed on Sep. 28, 2022.

(51) Int. Cl. A61K 31/4985; A61K 31/198; A61K 45/06; A61P 25/28

(52) U.S. Cl. CPC A61K 31/4985; A61K 31/198; A61K 45/06; A61P 25/28

(57) ABSTRACT

The present invention relates to a method of treating a mitochondrial disease in a patient in need thereof by administering Compound (I), a stimulator of soluble guanylate cyclase (sGC) at certain dosages either alone or in combination therapy.

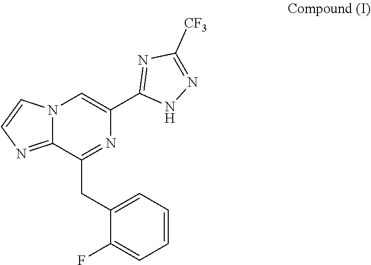 [SMILES: Fc1ccccc1Cc1nc(-c2nc(C(F)(F)F)n[nH]2)cn2ccnc12]

### RELATED APPLICATIONS

This application claims the benefit of priority to U.S. Provisional Application No. 63/350,708, filed on Jun. 9, 2022, and U.S. Provisional Application No. 63/410,829, filed on Sep. 28, 2022. The entire contents of each of the above-referenced applications are incorporated herein by reference.

### FIELD OF THE INVENTION

The present invention provides methods of treating certain mitochondrial diseases in human subjects by administering specific dosage regimens of a CNS-penetrant stimulator of soluble guanylate cyclase (sGC) either alone or in combination therapy.

### BACKGROUND OF THE INVENTION

### Mitochondrial Diseases and MELAS

Mitochondrial diseases or disorders are a group of rare genetic disorders that occur when mitochondria fail to produce enough energy for the body to function properly. They have clinically heterogeneous manifestations and they may manifest with impaired cerebral blood flow (CBF), oxidative stress, inflammation and metabolic crises, among other things. They can affect almost any part of the body, including the cells of the brain, nerves, muscles, kidneys, heart, liver, eyes, ears or pancreas. They cause debilitating physical, developmental, and cognitive disabilities with symptoms including poor growth, loss of muscle coordination, muscle weakness and pain, fatigue, seizures, vision and/or hearing loss, gastrointestinal issues, cognitive impairment, learning disabilities, and organ failure. Life expectancy in mitochondrial patients is greatly reduced. Mitochondrial disorders are usually progressive. It is estimated that 1 in 4,000 people has a mitochondrial disorder. 80% of patients with mitochondrial diseases display CNS symptoms.

Currently there is no effective treatment or cure for these disorders. Their management is mainly supportive therapy, which may include nutritional management, exercise and/or vitamin or amino acid supplements.

For example, mitochondrial encephalomyopathy, lactic acidosis, and stroke-like episodes (MELAS) is a particular mitochondrial disease that affects many of the body's systems, particularly the brain and nervous system (encephalo-) and muscles (myopathy). MELAS is the most common form of primary mitochondrial disease (https://www.mitoaction.org/resources/primary-mitochondrial-disease-and-secondary-mitochondrial-dysfunction-importance-of-distinction-for-diagnosis-and-treatment/). The signs and symptoms of this disorder most often appear in childhood following a period of normal development, although they can begin at any age. Early symptoms may include muscle weakness and pain, fatigue, recurrent headaches, loss of appetite, vomiting, and seizures. Most affected individuals experience stroke-like episodes (SLEs) beginning before age 40. These episodes often involve temporary muscle weakness on one side of the body (hemiparesis), altered consciousness, vision abnormalities, seizures, and severe headaches resembling migraines. Repeated SLEs can progressively damage the brain, leading to vision loss, problems with movement, and a loss of intellectual or cognitive function.

Most people with MELAS have a buildup of lactic acid in their bodies, a condition called lactic acidosis. Increased acidity in the blood can lead to vomiting, abdominal pain, extreme tiredness and fatigue, muscle weakness, and difficulty breathing. Less commonly, people with MELAS may experience involuntary muscle spasms (myoclonus), impaired muscle coordination (ataxia), hearing loss, heart and kidney problems, diabetes, and hormonal imbalances.

In the absence of approved therapies for MELAS, citrulline and L-arginine, precursors of nitric oxide (NO), are hypothesized to provide benefit in this patient population. The consensus guidelines from the Mitochondrial Medicine Society recommend acute arginine administration to improve clinical symptoms associated with SLEs in patients with MELAS. Mechanistically, L-arginine is converted directly into NO, the starting point of the nitric oxide-soluble guanylate cyclase-cyclic guanosine mono phosphate (NO-sGC-cGMP) pathway.

Treatment options for mitochondrial diseases remain extremely limited and, thus, there is still a need to develop new therapies that improve the many clinical manifestations associated with these diseases.

### SUMMARY OF THE INVENTION

In a first aspect of the invention, disclosed herein is a method of treating a mitochondrial disease in a patient by administering a total oral daily dose of Compound I of between 15 mg and 60 mg or an equal quantity in moles of a pharmaceutically acceptable salt of Compound I to said patient.

In a second aspect of the invention, disclosed herein is a Compound I or a pharmaceutically acceptable salt thereof for use in treating a mitochondrial disease in a patient by administering a total oral daily dose of Compound I of between 15 mg and 60 mg or an equal quantity in moles of a pharmaceutically acceptable salt of Compound I to said patient.

In a third aspect of the invention, disclosed herein is the use of Compound I or a pharmaceutically acceptable salt thereof for the manufacture of a medicament for treating a mitochondrial disease in a patient, by administering a total oral daily dose of Compound I of between 15 mg and 60 mg or an equal quantity in moles of a pharmaceutically acceptable salt of Compound I to said patient.

In a fourth aspect, the methods and uses of the invention involve treatment in combination with one or more additional therapeutic agents.

### BRIEF DESCRIPTION OF THE FIGURES

FIG. 1 shows a correlation of biomarkers of mitochondrial dysfunction GDF-15 and FGF-21 at baseline.

FIG. 2 shows an increase of CBF on day 29 when compared to baseline (day 1) observed across all brain regions of interest. CI=confidence interval.

### DETAILED DESCRIPTION OF THE INVENTION

### the NO-sGC-cGMP Pathway, sGC Stimulation and Mitochondrial Diseases

In the body, NO is synthesized from arginine and oxygen by various nitric oxide synthase (NOS) enzymes and by sequential reduction of inorganic nitrate. Three distinct isoforms of NOS have been identified: inducible NOS (iNOS or NOS II) found in activated macrophage cells; constitutive neuronal NOS (nNOS or NOS I), involved in neurotransmission and long-term potentiation; and constitutive endothelial NOS (eNOS or NOS III), which regulates smooth muscle relaxation and blood pressure.

sGC is the primary receptor enzyme for NO in vivo. sGC can be activated via both NO-dependent and NO-independent mechanisms. In response to this activation, sGC converts guanosine-5′-triphosphate (GTP) into the secondary messenger cyclic guanosine 3′, 5′-monophosphate (cGMP). The increased level of cGMP, in turn, modulates the activity of downstream effectors including protein kinases, phosphodiesterases (PDEs), and ion channels. Intracellular cGMP, by activating cGMP-dependent protein kinase (PKG) and other downstream modulators, regulates vascular tone and regional blood flow, fibrosis, and inflammation.

The NO signaling pathway is also critical for the regulation of mitochondrial function and mitochondrial biogenesis. NO pathway dysregulation is recognized as a major contributing factor in mitochondrial disease, and leads to impaired cerebral blood flow (CBF), oxidative stress, inflammation and metabolic crises. There are clear links observed between NO signaling, stroke-like episodes (SLEs), and dysregulated CBF in patients with mitochondrial disease. NO bioavailability may be reduced in these patients through several mechanisms, including endothelial dysfunction and concomitant reductions in endothelial NOS, increased levels of the NOS inhibitor asymmetric dimethylarginine (ADMA), and increases in oxidative stress and reactive oxygen species (ROS) that react with NO.

In the CNS, the NO-sGC-cGMP signaling pathway underlies multiple physiological processes that contribute to overall brain health, including neurotransmission, neurovascular function, cellular bioenergetics, and inflammation, and has been implicated in neuronal survival and cognitive function.

sGC stimulators are a class of heme-dependent agonists of the sGC enzyme that work synergistically with varying amounts of NO to increase its enzymatic conversion of GTP to cGMP. sGC stimulators are clearly differentiated from and structurally unrelated to another class of NO-independent, heme-independent agonists of sGC known as sGC activators. The benzylindazole compound YC-1 was the first sGC stimulator to be identified. Several sGC stimulators have been identified and pharmacologically characterized since then, including BAY 41-2272, BAY 41-8543, riociguat (BAY 63-2521), vericiguat, olinciguat (IW-1701), and praliciguat (IW-1973). No sGC stimulators have been approved for marketing in the field of CNS to date and to our knowledge, Compound I, depicted below, is the only CNS-penetrant sGC stimulator currently in clinical development for the treatment of CNS and mitochondrial diseases.

sGC stimulators may offer considerable advantages over other potential therapies that target the aberrant NO pathway or otherwise upregulate the NO pathway. For example, sGC stimulation is a more powerful approach than either the use of NO supplementation (which is associated with tachyphylaxis) or inhibition of cGMP breakdown (via phosphodiesterase inhibitors [PDEi]), which has limited effectiveness if cGMP levels are very low. In addition, the broad distribution of sGC, including in different areas of the brain, enables augmentation of signaling across tissues, while the PDEi targets have more limited cellular and tissue expression.

The consensus guidelines from the Mitochondrial Medicine Society recommend acute L-arginine administration to improve clinical symptoms associated with stroke like episodes in patients with MELAS. Mechanistically, L-arginine is converted directly into NO, the starting point of the NO-sGC-cGMP pathway. As a core node in the NO-sGC-cGMP pathway, it is hypothesized that an sGC stimulator is in a key position to potentially enhance mitochondrial function and biogenesis and have a positive effect in mitochondrial diseases, including those that affect the CNS.

### Compound I (CY6463)

Compound I (also named CY6463, IW-6463 or IWP-247) is an orally administered CNS-penetrant sGC stimulator being investigated for the treatment of CNS and mitochondrial diseases (clinical trials.gov identifiers NCT03856827, NCT04240158, NCT04475549, NCT04798989, NCT04972227). To our knowledge it is the only CNS-penetrant stimulator tested in human subjects to date.

As an sGC stimulator, Compound I acts as a positive allosteric modulator of sGC, by binding to sGC and thereby amplify downstream signaling.

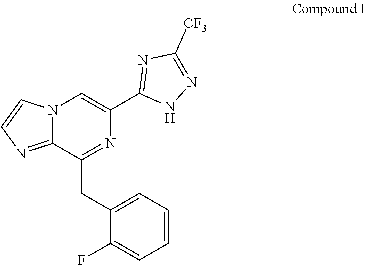 [SMILES: Fc1ccccc1Cc1nc(-c2nc(C(F)(F)F)n[nH]2)cn2ccnc12]

In vitro studies in mitochondrial disease patient cells indicated that Compound I may improve cellular energetics in these cells by increasing the abundance of available adenosine triphosphate (ATP) and may decrease mitochondrial dysfunction by increasing the expression of genes involved in mitochondrial function, ATP synthesis, metabolism, and ROS reduction (see WO2020/014504). Experiments in mitochondrial disease patient cells showed that expression levels of mitochondrial genes, such as TFAM and DDAH2 were lower in patient cells than in healthy cells. DDAH2 encodes for an enzyme that degrades asymmetric dimethylarginine (ADMA). TFAM is an abundantly expressed protein present in mitochondria that is necessary for mitochondrial transcription and regulates the mtDNA-copy number, thus being important for maintaining ATP production. ADMA increase can cause mitochondrial dysfunction and has been found to be elevated in mitochondrial disease patients. Consistent with increased ATP levels, treatment with Compound I increased expression levels of TFAM as well as DDAH2 in patient cells. In an in vivo model of mitochondrial dysfunction-induced retinal degeneration, mice pretreated with Compound I had lower rotenone-induced astrogliosis compared with vehicle-treated mice, indicating that Compound I may provide protection against tissue damage induced by mitochondrial dysfunction.

In rodent studies, a single dose of Compound I increased fMRI-BOLD signals, elevated qEEG gamma-band oscillatory power, and increased levels of cGMP in the CNS in contrast to a CNS-restricted sGC stimulator that demonstrated a lack of target engagement and distinct pharmacology in the CNS. In models of CNS impairment in rats, chronic dosing with Compound I improved dendritic spine density, reversed brain metabolite N-acetyl-aspartate (NAA)+N-acetylaspartate-glutamate (NAAG) deficits, restored hippocampal long term potentiation (LTP, a form of synaptic plasticity that underlies memory formation). Compound I also increased neurotrophic factors such as phosphorylated cAMP-response element binding (pCREB) and brain-derived neurotrophic factor (BDNF), and improved behavioral task performance in pharmacologically impaired rats (see Correia, Susana S; Iyengar, Rajesh R; Germano, Peter; Tang, Kim; Bernier, Sylvie G; Schwartzkopf, Chad D; Tobin, Jenny; Lee, Thomas W-H; Liu, Guang; Jacobson, Sarah; Carvalho, Andrew; Rennie, Glen R; Jung, Joon; Renhowe, Paul A; Lonie, Elisabeth; Winrow, C; Hadcock, J; Jones, J; Currie, MG. The CNS-Penetrant Soluble Guanylate Cyclase Stimulator CY6463 Reveals its Therapeutic Potential in Neurodegenerative Diseases. Front Pharmacol. 24 May 2021|https://doi.org/10.3389/fphar.2021.656561.)

Safety and pharmacokinetic (PK) data from a phase 1 study in healthy adults, together with safety, PK and pharmacodynamic (PD) data from a second phase 1 study in healthy elderly adults also supported clinical investigation of Compound I in the potential treatment of patients with mitochondrial disease in general, and MELAS in particular.

### Definitions and General Terminology

Unless otherwise defined, all technical and scientific terms used herein have the same meaning as commonly understood by one of ordinary skill in the art to which this invention belongs, which is the field of medicine, and of mitochondrial disease and/or brain medicine in particular. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the word “a” before a noun represents one or more of the particular noun. As used herein, the singular forms “a,” “an,” and “the” include plural referents unless the context clearly dictates otherwise.

As used herein, the terms “subject” and “patient” are used interchangeably. A subject or a patient is a human patient or human subject.

For the terms “for example” and “such as,” and grammatical equivalences thereof, the phrase “without limitation” or “and without limitation” is understood to follow unless explicitly stated otherwise.

Cognitive function naturally declines with age and also in pathological situations. “Cognitive impairment” refers to deficits in one or more higher brain functions that generally involve aspects of thinking and information processing (i.e., cognition).

The term “therapeutically effective amount” as used herein means that amount of active compound or pharmaceutical agent that elicits the medicinal response in a human that is being sought by a medical doctor or other clinician. The therapeutically effective amount of a compound is at least the minimum amount necessary to ameliorate, palliate, lessen, delay, reduce, alleviate, or cure a disease, disorder, or syndrome or one or more of its symptoms, signs or causes. In another embodiment, it is the amount needed to bring abnormal levels of certain clinical markers of the disease, disorder, or syndrome closer to the normal values or levels. In another embodiment, it is the amount needed to bring the levels of certain clinical markers displayed by a diseased subject closer to those of a normal subject of the same age (normalization) or closer to those of a patient with less severe disease presentation or that is in earlier stages of disease progression. An effective amount can be administered in one or more administrations throughout the day.

As used herein, a dose does not “result in a significant incidence of adverse events (AEs) or serious adverse events (sAEs) associated with symptomatic hypotension” if it does not result in excessive orthostatic hypotension, excessive dizziness, excessive postural dizziness, excessive pre-syncope, or excessive syncope in patients. Excessive orthostatic hypotension, excessive dizziness, excessive postural dizziness, excessive pre-syncope, or excessive syncope in patients are those that would warrant discontinuation of treatment by the patient or a recommendation of discontinuation by the practitioner.

The terms “administer”, “administering” or “administration” in reference to a compound or pharmaceutical agent, mean introducing the compound into the body of the patient in need of treatment. When Compound I or a pharmaceutically acceptable salt thereof is used in combination with one or more other therapeutic agents, “administration” and its variants are each understood to encompass concurrent and/or sequential introduction of Compound I and the other therapeutic agents into the patient.

The term “disorder”, as used herein refers to any deviation from or interruption of the normal structure or function of any body part, organ, or system that is manifested by a characteristic set of symptoms and signs and whose etiology, pathology, and prognosis may be known or unknown. The term disorder encompasses other related terms such as disease and condition (or medical condition) as well as syndromes, which are defined as a combination of symptoms resulting from a single cause or so commonly occurring together as to constitute a distinct clinical picture. In some embodiments, the term disorder refers to a mitochondrial disorder. As used herein the terms disorder, “disease”, “condition” or “syndrome” are used interchangeably.

“Mitochondrial disorders” refer to a group of genetic conditions that affect the mitochondria (the structures in each cell of the body that are responsible for making energy). These disorders can present at any age with almost any affected organ, including the brain, muscles, heart, liver, nerves, eyes, ears and kidneys. Some disorders affect only one organ or tissue, many involve multiple organ systems including the brain, muscles, heart, liver, nerves, eyes, ears and/or kidneys. Mitochondrial disorders have heterogeneous presentations.

Mitochondrial genetic disorders can be caused by mutations in either the mitochondrial DNA or nuclear DNA that lead to dysfunction of the mitochondria and inadequate production of cellular ATP. Those caused by mutations in mitochondrial DNA are transmitted by maternal inheritance, while those caused by mutations in nuclear DNA may follow an autosomal dominant, autosomal recessive, or X-linked pattern of inheritance. (See: https://rarediseases.info.nih.gov/diseases/7048/mitochondrial-genetic-disorders, last accessed Jun. 3, 2022, the teaching of which are incorporated herein by reference). Mitochondrial diseases contemplated throughout this disclosure are primary mitochondrial diseases or disorders. The term mitochondrial disorders as used here is equivalent with the term primary mitochondrial disorders. In some instances, mitochondrial dysfunction can also be secondary to other diseases. However treatment of such secondary mitochondrial dysfunction is not discussed or contemplated herein. See https://www.mitoaction.org/resources/primary-mitochondrial-disease-and-secondary-mitochondrial-dvsfunction-imnportance-of-distinction-for-diagnosis-and-treatment/ (last accessed 7 Jun. 2022) for definitions and distinctions between primary mitochondrial disorders or diseases and secondary mitochondrial dysfunction.

Mitochondrial diseases manifest primarily due to a chronic loss of cellular ATP that results in a variety of clinical phenotypes and symptomatology. In addition to the ATP crisis, mitochondrial respiratory chain dysfunction also causes excessive ROS production and increased oxidative stress, leading to cellular damage and inflammation.

Specific mitochondrial disease which may be treated and/or prevented by administering Compound I, or an equivalent amount of a pharmaceutically acceptable salt thereof, at the specific dosages here disclosed (total oral daily dose between 15 mg and 60 mg) include but are not limited to:

Alpers Disease, Autosomal Dominant Optic Atrophy (ADOA), Barth Syndrome/LIC (Lethal Infantile Cardiomyopathy), Beta-oxidation defects, Long Chain Fatty Acid Transport Deficiency, Co-Enzyme Q10 Deficiency, Complex I, II, III, IV, V Deficiency, Chronic Progressive External Ophthalmoplegia (CPEO), Friedreich's Ataxia, Kearns-Sayre syndrome, Leukodystrophy, Leigh Disease or Syndrome, LHON, LHON Plus, MELAS (Mitochondrial myopathy, encephalomyopathy, lactic acidosis, stroke-like symptoms), Myoclonic Epilepsy with Ragged Red Fibers (MERRF), Mitochondrial Recessive Ataxia Syndrome (MIRAS), Mitochondrial Cytopathy, Mitochondrial DNA Depletion, Mitochondrial Encephalopathy, Mitochondrial Myopathy, Multiple Mitochondrial Dysfunction Syndrome, MNGIE (Myoneurogenic gastrointestinal encephalopathy), NARP (Neuropathy, ataxia, retinitis pigmentosa, and ptosis), Pearson Syndrome, Pyruvate Carboxylase Deficiency, Pyruvate Dehydrogenase Deficiency or Pyruvate Dehydrogenase Complex Deficiency (PDCD/PDH), and POLG Mutations.

In one embodiment, the mitochondrial disease is selected from Alpers, Complex I, II, III, IV deficiency, CPEO, KSS, LCHAD, Leigh syndrome, Leukodystrophy, LHON, MELAS, MEPAN, MERRF, MIRAS, Mitochondrial DNA depletion, MNGIE, NARP, Pearson syndrome, and POLG mutations. In one embodiment, the mitochondrial disease is a Complex I mitochondrial disease. In another embodiment, the mitochondrial disease is MELAS. In yet another embodiment, the mitochondrial disease Leigh syndrome.

“Treat”, “treating” or “treatment” with regard to a disorder, disease, condition, symptom or syndrome, refers to abrogating or improving the cause and/or the effects (i.e., the symptoms, physiological, physical, psychological, cognitive, emotional or functional manifestations, or any of the clinical parameters or observations) associated with the disorder, disease, condition or syndrome. As used herein, the terms “treat”, “treatment”, and “treating” also refer to the delay or amelioration or slowing down or prevention of the progression (i.e., the known or expected progression of the disease), severity, and/or duration of the disease or delay or amelioration or slowing down or prevention of the progression of one or more clinical parameters associated with the disease (i.e., “managing” without “curing” the condition), resulting from the administration of one or more therapies.

The phrase “pharmaceutically acceptable salt,” as used herein, refers to pharmaceutically acceptable organic or inorganic salts of Compound I. The pharmaceutically acceptable salts of Compound I may be used in medicine. Salts that are not pharmaceutically acceptable may, however, be useful in the preparation of Compound I or of other Compound I pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of Compound I described herein include those derived from Compound I with inorganic acids, organic acids or bases. In some embodiments, the salts can be prepared in situ during the final isolation and purification of the compounds. In other embodiments the salts can be prepared from the free form of Compound I in a separate synthetic step.

When a compound such as Compound I is acidic or contains a sufficiently acidic moiety, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N, N′dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When a compound such as Compound I is basic or contains a sufficiently basic moiety, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66:1-19, incorporated here by reference in its entirety.

### Clinical Assessments and Patient-Reported Outcome Tools

The assessment of health status in a patient with mitochondrial disease and the assessment of the corresponding pathology underlying the observed dysfunction, decline, or symptoms, may be carried out using a number of different assessment tools or clinical measurements known and used in the field.

These range from imaging tools (e.g., magnetic resonance imaging (MRI), such as using arterial spin labeling (ASL) or functional fMRI-BOLD modalities), to laboratory measurements (e.g., fluid biomarkers measured in blood, cerebro-spinal-fluid (CSF), urine, plasma, serum, skin, saliva), to clinical outcome assessment tools or instruments (e.g., patient- or clinician-reported outcome instruments or performance outcome measures, for instance cognitive assessments using PROMIS questionaries, MFIS scoring and others described herein or known in the art), digital assessments (e.g., those obtained with wearable devices, sensor- or camera-based assessments) and electrophysiological assessments (e.g., EEG). Some of these are described in the Examples section below and were used in the clinical trial described in Example 1. Others are known in the art and could be used in the hospital, clinical or community setting. For example, the American Association of Family Physicians (AAFP), in its webpage, describes and provides links to a number of potential cognitive assessment tools, such as MiniCog, MoCA, SLUMS Examination, CPCoG, MIS and MMSE and others (https://www.aafp.org/pubs/afp/issues/2019/0115/p101.html, last accessed on 3 Jun. 2022).

Some measurements are carried out to help in diagnosis and or patient selection. Others are carried out to help in assessing prognosis. Others may be carried out to assess pharmacological responses to a certain intervention (pharmacodynamic or PD assessments) such as described herein. Others may be carried out to assess susceptibility to or risk of decline or response to a certain intervention (e.g., assessment of genetic markers or other biomarkers) or to assess disease progression in a patient.

Prior Clinical Data with Compound I in Healthy Subjects

Electroencephalography (EEG) is a technique that measures electrical activity in the brain (brain electrophysiology) by using electrodes places on the scalp. EEG-power spectral signals may be analyzed at different frequencies or frequency bands. They were analyzed at the following frequency bands in two prior phase 1 clinical trials with Compound I described below: Delta-1-4 Hz (typically associated with sleep), Theta-4-7.5 Hz (associated with waking/falling asleep, some association with cognition), Alpha-8-12 Hz (associated with passive wakefulness, and with cognitive processing), Beta-12-25 Hz (associated with alertness and concentration) and Gamma-25-45 Hz (associated with higher cognitive function). qEEG stands for quantitative electroencephalography.

An event-related potential (ERP) is “a time-locked measure of electrical activity of the cerebral surface representing a distinct phase of cortical processing” for example in response to an auditory or visual stimulus (Patel and Azzam (2005), Characterization of N200 and P300: Selected Studies of the Event-Related Potential. International Journal of Medical Sciences 2(4):147-154). ERPs are time-locked and represent the average of the electrical responses observed after multiple attempts. ERPs are an objectively non-invasive approach for studying information processing and cognitive functions in the brain.

During EEG-ERP experiments, several key waveforms may be evaluated following a stimulus: N200 (associated with stimulus identification and distinction), P300 (associated with selective attention, information processing and cognitive speed/capacity), P200 (associated with aspects of selective attention or stimulus encoding), P50 (assciated with sensory gating, or the reduced neurophysiological response to redundant stimuli), and N100 (associated with early perceptal processes).

Two key parameters are used to quantify each response: latency (how long after the stimulus is the peak signal) and amplitude (how strong is the peak signal).

In pre-clinical models, sGC stimulation by Compound I had been shown to lead to changes of qEEG signals (see Meeting abstracts from the 9th International Conference on cGMP: Generators, Effectors and Therapeutic Implications, Journal of Translational Medicine volume 17, Article number: 254 (2019) S 1-02 Evaluating soluble guanylate cyclase stimulation for serious central nervous system diseases; Correia, Susana S; Iyengar, Rajesh R; Germano, Peter; Tang, Kim; Bernier, Sylvie G; Schwartzkopf, Chad D; Tobin, Jenny; Lee, Thomas W-H; Liu, Guang; Jacobson, Sarah; Carvalho, Andrew; Rennie, Glen R; Jung, Joon; Renhowe, Paul A; Lonie, Elisabeth; Winrow, C; Hadcock, J; Jones, J; Currie, MG. The CNS-Penetrant Soluble Guanylate Cyclase Stimulator CY6463 Reveals its Therapeutic Potential in Neurodegenerative Diseases. Front Pharmacol. 24 May 2021|https://doi.org/10.3389/fphar.2021.656561). These studies were performed in rats with telemetry devices implanted in the frontal cortical and front-parietal regions of the brain. Rats were dosed with a suspension of Compound I orally, a suspension of a peripherally restricted sGC stimulator orally, or a solution of donepezil by subcutaneous injection. Compound I altered qEEG measurements including increasing gamma oscillations while the peripherally restricted sGC stimulator reduced gamma power compared to vehicle dosing. Compound I given to rats orally at 10 mg/kg increased gamma power and the signal was further increased in combination with 1 mg/kg donepezil at 1-2 hours post-dose.

Subsequently, in a Phase I clinical trial (clinical trial.gov identifier NCT03856827) changes were also observed in the brains of healthy participants aged 18 to 62 by EEG after QD dosing with 15 mg of Compound I. More specifically, in that study effects of Compound I on ERP P300 amplitude were observed, with an increase in amplitude with increasing dose level, as well as responses being modulated by time since dosing. Improvements in alpha power were also observed in the NCT03856827 study at day 14, across all dosage levels tested and compared to placebo.

A second Phase I clinical trial (clinical trial.gov identifier NCT04240158) was run with elderly subjects [described in a prior patent application publication (WO 2022/081610) and in A Phase 1 Translational Pharmacology Study in Healthy Elderly Volunteers Evaluating the Safety, Tolerability, Pharmacokinetics, and CNS Activity of IW-6463, a CNS-penetrant, Soluble Guanylate Cyclase Stimulator, Chad Glasser, Jacob Donoghue, Phillip Alday, Alex Arslan, Emily Florine, Chris Winrow, Chris Wright Neurology April 2021, 96 (15 Supplement) 4701)]. In this trial increases in posterior alpha power, trend increases in gamma power, and shortening of N200 auditory event-related potential (ERP) latencies were observed via EEG. Improvements in saccadic reaction time and saccadic peak velocity in saccadic eye movement (SEM) assessments were also observed. Furthermore, in an exploratory CSF biomarker analysis, positive trends on important CSF neuroinflammatory markers were observed in subjects after two weeks of treatment with 15 mg of Compound I QD as compared with placebo. In particular, potentially important reductions in the concentrations of Alpha 2 macroglobulin (A2M) and complement C3 (C3) biomarkers were observed. The larger reductions in concentrations of these biomarkers were observed in subjects older than 70 years old. A2M elevations are related to cerebrovascular disease and predict cognitive decline and development of AD. They have been reported to lead to tau hyperphosphorylation. C3 is associated with Aβ and tau and may play a role in synaptic degeneration.

In addition, several functional neuroimaging techniques were used in the NCT04240158 trial in healthy elderly participants: MRI-Arterial spin labelling (MRI-ASL) and functional magnetic resonance imaging (fMRI). The neuronal metabolic profile of the brain (as an additional indicator of cellular bioenergetics) was also measured by magnetic resonance spectroscopy (1H-MRS).

MRI-ASL is used to quantify regional CBF during the resting state. fMRI is a relative measure based on the BOLD effect, described below, and it is used to measure the change in CBF in certain regions of the brain as a result of increased brain activity.

The BOLD effect is based on the fact that blood flow in the brain is highly locally controlled in response to oxygen and carbon dioxide tension of the cortical tissue. When a specific region of the cortex increases its activity in response to a task, the extraction fraction of oxygen from the local capillaries leads to an initial drop in oxygenated hemoglobin (oxyHb) and an increase in local carbon dioxide (CO2) and deoxygenated hemoglobin (deoxyHb). Following a lag of 2-6 seconds, CBF increases, delivering a surplus of oxygenated hemoglobin, washing away deoxyhemoglobin. It is this large rebound in local tissue oxygenation which is imaged. The reason fMRI is able to detect this change is due to a fundamental difference in the paramagnetic properties of oxyHb and deoxyHb. The NeuroCart® battery of assessments was also run in the NCT04240158 trial with healthy elderly subjects. NeuroCart® is a full battery of tests for measuring a wide range of CNS functions that was developed by the Center of Human Drug Research (CHDR). NeuroCart can be used to correlate a compound's CNS effects with drug concentration, helping determine whether an effect is due to the compound specifically. NeuroCart provides both objective (e.g. neurophysiology, brain performance) and subjective (e.g. cognitive function, memory, mood, etc.) measures of CNS function. NeuroCart included EEG, and SEM testing in addition to a diverse set of other assessments of CNS function as described in WO 2022/081610.

In the NCT04240158 trial, positive effects were observed in EEG and SEM assessements which support a beneficial effect of Compound I in some aspacts of cognition and the potential use for the treatment of cognitive impairment is patients in need thereof as described in WO 2022/081610. No changes were observed in neuroimaging assessments by ASL and fMRI BOLD, or MRS measurements, as well as on NeuroCart CNS function assessments in that trial. Results on EEG and SEM of that trial supported the treatment of cognitive impairment or decline in patients suffering from a diverse set of diseases, including mitocondrial diseases.

Compound I in Patients with MELAS

In the two prior phase 1 clinical trials above described, changes in EEG paramenters (alpha power and N200 ERP), changes in SEM, as well as a tendency towards improvement in two neuroinflammation parameters were observed in healthy elderly subjects treated with multiple dosing of 15 mg QD for 14 days.

A goal of the study described in the Examples section of this disclosure (Example 1) was to assess the effect of a daily oral dose of 15 mg of Compound I (QD) on a number of parameters or measures related to brain and general health, in patients with the mitochondrial disease MELAS. The enrolled patients had genetically confirmed MELAS with a history of CNS symptoms such as headache, seizure and stroke. They were allowed to be on stable medications including NO precursors (e.g., arginine and citrulline).

The different assessments and measurements carried out are described in detail in the Examples section.

The primary objective of the trial was to evaluate the safety and tolerability of Compound I when administered to a patient population of subjects with MELAS, by measuring adverse events in the form of AEs, SAEs and TEAEs (defined in the experimental section) leading to drug discontinuation.

Exploratory objectives included to evaluate the plasma PK of the compund. In addition, other exploratory endpoinds were aimed at evaluating the effect of Compound I on physiology, neurophysiology and cognitive and health status. In particular, changes from baseline in plasma biomarker concentrations of a number of relevant biomarkers of inflammation and biomarkers linked to mitochondrial dysfunction on day 29, change from baseline in CBF measured by ASL on day 29, changes from baseline in fMRI-BOLD signals during resting state and a visual stimulus on day 29, change from baseline in brain metabolite levels by 1H-NMR on day 29, change from baseline in MFIS scores (total, physical, cognitive and psychosocial subscores) on day 29 and change from baseline in PROMIS Item Bank v2.0—cognitive function total score on day 29 were measured.

The present invention is based on the surprising finding that Compound I, administered at a total oral daily dosage of 15 mg per day, to a population of patients with the mitochondrial disease MELAS was safe and well tolerated and showed evidence of impacts on CBF, fMRI-BOLD as well as on biomarkers of mitochondrial dysfunction and a large number of inflammatory biomarkers. The biomarkers of mitochondrial dysfunction studied are known biomarkers of bioenergetics and metabolism which have been found to be elevated in mitochondrial disease patients. CBF and fMRI-BOLD are neuroimaging measures of brain perfusion and neuronal function/connectivity, respectively.

Inflammatory processes in patients with mitochondrial dysfunction are known to be upregulated, both peripherally and centrally. Improvements across the inflammation panel described below suggest Compound I has positive impacts on oxidative stress.

Neuronal and/or glial injury due to mitochondrial failure, nitric oxide deficiency and cerebrovascular angiopathy impact cerebral perfusion (CBF). Dysregulated cerebral perfusion is linked to stroke-like episodes and CNS symptoms.

In addition, improvements in patients' perceived health status, fatigue and cognition assessments were observed for several of the subjects in the trial and some of these improvements were consistent with improvements in CBF, fMRI and biomarkers in those same patients.

Therefore, results of the trial described in Example 1 in the Examples section demonstrated positive effects across multiple biomarkers, patient-reported outcomes, increased cerebral blood flow, increased functional connectivity within neural networks associated with cognition, memory and executive function.

Therefore, Compound I has the potential to be used to treat additional aspects of mitochondrial disease, including the physical aspects of the disease, and to improve the general health status of the patient in addition to the potential of improving some aspects of cognition as previously reported.

The results of this trial with Compound I support the potential to leverage sGC stimulation to benefit several key pathological mechanisms of mitochondrial disease and dysfunction by using specific dosage regimens of Compound I in human subjects. These include effects on cellular bioenergetics, inflammation, neuronal/cognitive function and impaired brain perfusion or cerebral blood flow. In particular, the fact that known biomarkers of mitochondrial dysfunction such as lactate levels, and FGF-21 and GDF-15, that were elevated at baseline, were reduced, and that such reduction is correlated with Compound I plasma levels, supports the potential for Compound I to be a disease-modifying treatment that can improve many aspects of the mitochondrial disease phenotype or presentation. In addition, it has the potential to work across other mitochondrial diseases displaying related symptoms, in addition to MELAS. Importantly, the largest improvements were observed in the patients with the more severe disease phenotypes at baseline.

Multiple plasma inflammatory and biomarkers of mitochondrial dysfunction were found to be elevated at baseline across participants and multiple plasma inflammatory and biomarkers of mitochondrial dysfunction demonstrated directional improvement with treatment, many of which correlated with plasma exposures of Compound I (i.e. higher exposures at the end of treatment, corresponded to higher improvement in biomarkers).

Fibroblast growth factor-21 (FGF-21) is a hormone-like cytokine that is involved in intermediary metabolism of carbohydrates and lipids. FGF-21 expression is driven by mitochondrial reactive oxygen species and concentrations of FGF-21 are known to be drastically higher in mitochondrial disease patient compared to controls.

Growth differentiation factor 15 (GDF-15) is a member of the transforming growth factor beta family and was first selected as a marker for mitochondrial dysfunction via a gene expression study of skeletal muscle from patients with mitochondrial disease caused by a Thymidine Kinase 2 mutation compared to healthy skeletal muscle. They found that GDF-15 was significally upregulated in both skeletal muscle and serum of patients with mitochondrial dysfunction. It is known that GDF-15 expression can be induced in response to stress such as mitochondrial dysfunction via upregulation of the activating transcription factor 4.

Lactate is a product of the anaerobic production of ATP and is the most commonly used marker to detect mitochondrial dysfunction in the general diagnosis of MD. Although not particularly sensitive (between 34 and 62% sensitivity), elevated lactate concentrations have an estimated specificity between 83 and 100% to detect mitochondrial disease.

CBF increased with treatment across participants in all regions of interest and was consistent with patient global impression of change (PGIC) (i.e. better PGIC was associated with larger increases in CBF), and with improvements in inflammatory biomarker concentrations.

fMRI BOLD signal observed during both resting state and with a visual task indicated increased functional connectivity within neural networks associated with cognition, memory, executive function and sensorimotor processing with treatment, and was consistent with improvements in CBF. fMRI-BOLD response to visual stimulus is known to be markedly reduced in symptomatic MELAS compared to controls (Rodan et al 2020). Compound I treatment increased activation of occipital brain regions in response to the visual stimulus, with greater activation at Day 29 compared to Day 1

Trends towards improvement in the PGIC were observed although clinical outcomes of fatigue (MFIS) and cognition (PROMIS) did not indicate improvements with treatment at the study level. However, in several patients responding more poorly on these assessments at baseline, improvements were observed. Improvements in patient-reported outcomes, including fatigue, cognition, and overall disease perception were reported in patients with most severe disease.

Therefore pharmacodynamic signals were observed across four disease domains: CBF (measured through ASL), inflammation (measured through plasma inflammatory biomarker levels), cellular bioenergetics (measured through mitochondrial dysfunction biomarker levels associated with metabolism and bioenergetics) and neuronal function and connectivity (measured by fMRI and cognitive patient-reported outcome assessments) in this trial.

Furthermore, in another clinical trial in patients with high levels of pathology (patients with stable schizophrenia) that run concurrently with the MELAS trial, it was also discovered that total oral daily dosages up to 60 mg per day produce proportional PK and are safe and well tolerated. Thus, it is highly plausible that dosages up to 60 mg per day are also safe and well tolerated in mitochondrial disease patients and they have the potential to lead to even higher pharmacological responses to those herein described.

In addition, even though the trial was run in patients with MELAS, the fact that positive effects were observed at the study level, despite the high heterogeity of the population enrolled, make it highly plausible that a similar positive response could be observed in other mitochondrial disease patients that present with similar phenotyes, or for which disease is manifested as a result of similar mechanisms.

### Therapeutic Methods/Embodiments

In some embodiments of the methods and uses of the invention, a therapeutically effective amount of Compound I is a total oral daily dose of between 15 and 60 mg of Compound I. In some embodiments, it is a total oral daily dose of 15 mg. In other embodiments, it is a total oral daily dose of 20 mg. In other embodiments it is a total oral daily dose of 25 mg. In still other embodiments, it is a total oral daily dose of 30 mg. In still some embodiments, it is a total oral daily dose of 45 mg. In yet other embodiments, it is a total oral daily dose of 60 mg.

In some embodiments, a pharmaceutically acceptable salt of Compound I can be used in the methods and uses of the invention described herein. When a pharmaceutically acceptable salt of Compound I is used, the dose for the pharmaceutically acceptable salt depends on the molecular weight of the salt and has an equal quantity in moles to the dose of Compound I described herein. Accordingly, in some embodiments, the present invention is a method of treating a patient with a mitochondrial disease by administering a total oral daily dose of Compound I of between 15 mg and 60 mg or an equal quantity in moles of a pharmaceutically acceptable salt of Compound I to said patient.

In some embodiments, Compound I is indicated for the treatment of mitochondrial diseases. In some embodiments, the mitochondrial diseases that may be most suitable for treatment with Compound I include, but are not limited to, those that present with a similar phenotype to MELAS. Mitochondrial disease patients with phenotypes similar to MELAS include those with a confirmed mitochondrial disease mutation and at least two of the following: a history of at least one SLE; a history of at least one encephalopathic episode defined as one or more episodes of personality or behavioral change, confusion or disorientation in time, place or person; a history of symptomatic seizures; a history of migraine headaches preventing the individual from functioning normally at school, work or at home occurring on average at least one day per month during the last 3 months; or cognitive impairment defined as consistent forgetfulness with partial recollection of events, memory loss and/or difficulty problem solving.

In some embodiments of the above methods and uses of the invention, Compound I or a pharmaceutically acceptable salt thereof is indicated for the treatment of patients with a mitochondrial disease selected from:
Alpers Disease, Autosomal Dominant Optic Atrophy (ADOA), Barth Syndrome/LIC (Lethal Infantile Cardiomyopathy), Beta-oxidation defects, Co-Enzyme Q10 Deficiency, Complex I, II, III, IV, V Deficiency, Chronic Progressive External Ophthalmoplegia (CPEO), Friedreich's Ataxia, Kearns-Sayre syndrome, Leukodystrophy, Leigh Disease or Syndrome, LHON, LHON Plus, MELAS (Mitochondrial myopathy, encephalomyopathy, lactic acidosis, stroke-like symptoms), Myoclonic Epilepsy with Ragged Red Fibers (MERRF), Mitochondrial Recessive Ataxia Syndrome (MIRAS), Mitochondrial Cytopathy, Mitochondrial DNA Depletion, Mitochondrial Encephalopathy, Mitochondrial Myopathy, Multiple Mitochondrial Dysfunction Syndrome, MNGIE (Myoneurogenic gastrointestinal encephalopathy), NARP (Neuropathy, ataxia, retinitis pigmentosa, and ptosis), Pearson Syndrome, Pyruvate Carboxylase Deficiency, Pyruvate Dehydrogenase Deficiency or Pyruvate Dehydrogenase Complex Deficiency (PDCD/PDH), and POLG Mutations.

In some embodiments, the mitochondrial disease is selected from Alpers, Complex I, II, III, IV deficiency, CPEO, KSS, LCHAD, Leigh syndrome, Leukodystrophy, LHON, MELAS, MEPAN, MERRF, MIRAS, Mitochondrial DNA depletion, MNGIE, NARP, Pearson syndrome, and POLG mutations.

In some embodiments, the mitochondrial disease is a Complex I mitochondrial disease. In some embodiments, the mitochondrial disease is MELAS. In other embodiments, the mitochondrial disease is Leigh syndrome.

In some embodiments of the above methods and uses of the invention, treatment with Compound I or a pharmaceutically acceptable salt thereof does not result in an adverse event (AE) or serious adverse event (SAE) associated with excessive symptomatic hypotension or orthostatic hypotension.

In some embodiments of the methods and uses of the invention, the human patient is between 16 and 75 years old. In other embodiments, the patient is between 16 and 70 years old. In other embodiments, the patient is between 16 and 65 years old. In still other embodiments, the patient is between 16 and 60 years old. In some embodiments, the patient is between 16 and 55, between 16 and 50, between 16 and 40, or between 16 and 30 years old.

In some embodiments, the human patient is 16 years or older. In other embodiments, the human patient is 18 years or older. In still other embodiments, the patient is younger than 65 years old, younger than 60 years old, younger than 50 years old, younger than 40 years old, younger than 30 years old or younger than 20 years old. In still other embodiments, the patient is a child. In yet other embodiments, the patient is an adult. In still other embodiments, the patient is an adolescent. In still other embodiments, the patient is younger than 16 years old. In other embodiments, the patient is 12 years or older. In some embodiments, the patient is 3 years or older. In still other embodiments, the patient is 12 years old or younger. In other embodiments, the patient is 10 years older or younger. In other embodiments, the patient is 5 years old or younger. In some embodiments, the patient is between 3 and 18 years old, between 3 and 12 years old, between 5 and 18 years old, between 5 and 12 years old, or between 3 and 5 years old.

In some embodiments of the methods and uses of the invention, the human patient has been treated with one or more other therapeutic agent used for treating mitochondrial disease prior to the treatment with Compound I or a pharmaceutically acceptable salt thereof. In one embodiment, the other therapeutic agent used for treating mitochondrial disease is selected from citrulline and arginine. In another embodiment, the other therapeutica agent is a mito cocktail as described herein.

In some embodiments of the above methods and uses, the total oral daily dose is given as a single dose (QD). In other embodiments, the total oral daily dose can be split into two equal oral daily dosages (BID) of between 7.5 mg and 30 mg.

In certain embodiments, the methods and uses of the present invention described herein comprise administering to the patient a single oral daily dose of 15 to 60 mg of Compound I or an equal quantity in moles of a pharmaceutically acceptable salt of Compound I.

In certain embodiments, the methods and uses of the present invention described herein comprise administering to the patient a single oral daily dose of 15 mg of Compound I or an equal quantity in moles of a pharmaceutically acceptable salt of Compound I.

In certain embodiments, the methods and uses of the present invention described herein comprise administering to the patient a single oral daily dose of 20 mg of Compound I or an equal quantity in moles of a pharmaceutically acceptable salt of Compound I.

In certain embodiments, the methods and uses of the present invention described herein comprise administering to the patient a single oral daily dose of 25 mg of Compound I or an equal quantity in moles of a pharmaceutically acceptable salt of Compound I.

In certain embodiments, the methods and uses of the present invention described herein comprise administering to the patient a single oral daily dose of 30 mg of Compound I or an equal quantity in moles of a pharmaceutically acceptable salt of Compound I.

In certain embodiments, the methods and uses of the present invention described herein comprise administering to the patient a single oral daily dose of 45 mg of Compound I or an equal quantity in moles of a pharmaceutically acceptable salt of Compound I.

In certain embodiments, the methods and uses of the present invention described herein comprise administering to the patient a single oral daily dose of 60 mg of Compound I or an equal quantity in moles of a pharmaceutically acceptable salt of Compound I.

In some embodiments, the methods and uses of the invention described herein comprise administering an initial total oral daily dose of 30 to 60 mg of Compound I or an equal quantity in moles of a pharmaceutically acceptable salt of Compound I to the patient followed by a down-titration to a total oral daily dose of 15 to 30 mg of Compound I or an equal quantity in moles of a pharmaceutically acceptable salt of Compound I if the patient does not tolerate above 30 mg daily dose as assessed by a medical practitioner. Sometimes, the patient can go back to a higher dose after a period of time adapting to a lower dose, once that lower dose has been tolerated for a relevant period of time, as assessed by a medical practitioner.

In certain embodiments, the methods and uses of the present invention described herein comprise administering to the patient an oral dose of 7.5 to 30 mg of Compound I or an equal quantity in moles of a pharmaceutically acceptable salt of Compound I twice a day (BID). In one embodiment, the methods and uses of the present invention described herein comprise administering to the patient an oral dose of 7.5 mg, 10 mg, 12.5 mg, 15 mg, 22.5 mg or 30 mg of Compound I or an equal quantity in moles of a pharmaceutically acceptable salt of Compound I twice a day (BID). In one embodiment, the methods and uses comprise administering to the patient a first oral dose of 7.5 to 30 mg (e.g., 7.5 mg, 10 mg, 12.5 mg, 15 mg, 22.5 mg or 30 mg of Compound I) or an equal quantity in moles of a pharmaceutically acceptable salt of Compound I and a second oral dose of 7.5 to 30 mg (e.g., 7.5 mg, 10 mg, 12.5 mg, 15 mg, 22.5 mg or 30 mg of Compound I) or an equal quantity in moles of a pharmaceutically acceptable salt of Compound I, wherein the first dose and the second dose are separated by a period between 5 hours and 15 hours, between 8 hours and 15 hours, or between 10 hour and 15 hours. In another embodiment, the first dose and the second dose are separated by 5 hours, 6 hours, 7 hours, 8 hours, 9 hours, 10 hours, 11 hours, 12 hours, 13 hours, 14 hours, or 15 hours.

In some embodiments, the maintenance dose continues indefinitely as long as the patient continues to experience clinical benefit. In some embodiments, the treatment with Compound I is chronic.

In some embodiments of the above methods and uses, Compound I is administered before a symptom of mitochondrial disorder fully develops in said patient. In other embodiments of the above methods and uses, Compound I is administered after one or more symptoms of mitochondrial disorder develops in said patient.

In some embodiments, the patient with a mitochondrial disorder is one who has been diagnosed with it or who is genetically predisposed to the development of said disorder. In other embodiments a patient in need thereof is a person that has been genetically tested and found to have a mutation in a gene that predisposes him or her to the development of said disorder, even though he or she may not show any physical symptoms of the disorder (or disease) yet, or may show minimal symptomatology. In still other embodiments, the patient displays symptoms of the disorder (or disease) even though a formal diagnosis has not been made yet. Some common signs and symptoms of mitochondrial disorder include: poor growth, loss of muscle coordination, muscle weakness, fatigue, exercise intolerance, lactic acidosis, seizures, stroke-like episodes (SLEs), headaches, cognitive impairment, mental fatigue, fatigue, autism, problems with vision and/or hearing, developmental delay, learning disabilities, heart, liver, and/or kidney disease, gastrointestinal disorders, diabetes, increased risk of infection, thyroid and/or adrenal abnormalities, autonomic dysfunction, and dementia.

In some embodiments, treatment results in amelioration of at least one measurable physical parameter of a mitochondrial disorder. In other embodiments, treatment results in the reduction, inhibition or slowing down of the progression of a mitochondrial disorder either by, e.g., stabilization of a measurable symptom or set of symptoms, or by, e.g., stabilization of a measurable biomarker.

In some embodiments, treatment results in a measurable improvement in neuronal function and connectivity. In one embodiment, the improvement in neuronal function and connectivity is measured by functional magenic resonance imaging (fMRI).

In some embodiments, treatment results in an increase in cerebral blood flow (CBF). In one embodiment, treatment results in an increase in cerebral blood flow (CBF) in a brain region selected from temporal lobe, parietal lobe, occipital lobe, frontal lobe, corpus callosum, cingulate lobe, cerebral white matter and cerebellar white matter, and a combination of one or more aforementioned regions.

In some embodiments, treatment results in a reduction in one or more inflammatory biomarkers. In some embodiments, the inflammatory biomarkers described herein are selected from VCAM-1 (vascular cell adhesion molecule-1), ICAM (intercellular adhesion molecule), vWF (von Willebrand factor), and TNFR2 (tumor necrosis factor receptor 2). In other embodiments, treatment results in a reduction of one or more biomarkers of mitochondrial dysfunction. In still other embodiments, treatment results in reduction in one or more inflammatory biomarkers and one or more biomarkers of mitochondrial dysfunction. In some embodiments, the biomarkers of mitochondrial dysfunction described herein are selected from lactate, GDF-15 and FGF-21.

### Combination Therapies

The treatment of mitochondrial diseases and related symptoms with Compound I or a pharmaceutically acceptable salt thereof can be carried out using the compound alone or in combination therapy with other therapeutic agents. In some embodiments, Compound I or a pharmaceutically acceptable salt thereof can be used for the treatment of mitochondrial diseases in combination with one or more medications independently selected from citrulline and arginine.

In some embodiments, Compound I or a pharmaceutically acceptable salt thereof can be used for the treatment of mitochondrial diseases in combination with a mito cocktail. As used herein, a “mito cocktail” refers to a combination of a variety of vitamins and supplements which are commonly used by adults and children who have been diagnosed with mitochondrial disease.

The supplements and vitamins used by mitochondrial disease patients are often high doses and could require a patient to take up to 50 different pills per day. A compounding pharmacist through the International Academy of Compounding Pharmacists (IACP), can create a “cocktail” in a liquid, capsule or other form that combines the pure powdered form of the prescribed supplements and vitamins. The final medication is usually a much smaller amount than if otherwise taken, and can even be flavored to improve palatability. By avoiding fillers common in over the counter tablets, an individual's allergy or dietary restrictions can be accommodated. The exact compound, including dosage and ingredients, is determined by the patient's physician and differs depending on an individual patient's diagnosis, clinical symptoms, and weight. The most common ingredients include the following:

### 1) Coenzyme Q-10

Coenzyme Q-10 (Coenzyme Q10, CoQ10, CoQ-10, CoQ, ubiquinone, Q-Gel®), is a fat-soluble vitamin-like substance present in every cell of the body and serves as a coenzyme for several of the key enzymatic steps in the production of energy within the cell. It also functions as an antioxidant protecting against accumulation of harmful free radicals, which is important in its clinical effects. Many patients report increased energy while using Coenzyme Q-10, and thus it is a common “front-line” approach to supporting children and adults with mitochondrial disease. Frequently reported side effects include stomach upset and sleep disturbance, so the pharmacists recommend taking Co Q-10 doses earlier in the day and with food. Therapeutic levels may need time to be established, so patients may not see an immediate beneficial effect. In addition, the excess of the substance that is not used is stored in the fat cells, so proper dosing is important.

### 2) Complex Vitamins

Some B-vitamins are cofactors which participate in important mitochondrial reactions. Most of the B-vitamins have a bitter taste and more palatable if flavored. B-vitamins are water soluble; that is, they are excreted if not used, and the benefit from taking these vitamins should be felt immediately.
a. Vitamin B1 (Thiamine). This is a water soluble vitamin which helps break down carbohydrates so the body can better use them, helps with growth and maintenance of muscle tone, and aids memory. The only possible side effect sometimes noted is drowsiness.
b. Vitamin B2 (Riboflavin). Also a water soluble vitamin, B2 is necessary for energy production in the mitochondria and increases muscle performance as well as helping maintain healthy mucous membranes, skin, hair and nails. The only side effect noted is the tendency to turn urine an orange color. Given in the form of Riboflavin Biphosphate can improve the taste of this vitamin.
c. Vitamin B3 (Niacin). Occasionally used, B3 can often cause flushing of the face so it is generally given separately first to see if any side effects will occur before it is added to the cocktail.
d. Vitamin B6 (Pyridoxine) and Vitamin B12 (Cobalamine). These are other B vitamins which are frequently part of the compounded mix which patients with Mito may use.
e. Vitamin C. This is used for its help in the healing process and to ward off infections but can cause some stomach upset and occasionally headaches when the dose is increased.
f. Vitamin E. This protects cell membranes and improves neurological function. Usually the dose is no higher than 400-600 mg per day for an adult. Vitamine E can interfere with coumadin/warfarin medications and caution for the use of Vitamine E in mito cocktail is warranted.
g. Vitamin K1. Another vitamin that may be added (but with caution as there is a very small safe range for the dosage of this vitamin) must be prescribed by a physician, and is not to be purchased over the counter.

### 3) Other Antioxidants

Antioxidants decrease free radical accumulation in the cells and therefore are used for mitochondrial disease patients as well. Alpha Lipoic Acid is probably the most commonly prescribed anti-oxidant used in the Mito cocktail.

### 4) L-Carnitine

L-Carnitine helps transport fatty acids and improve the strength and tone of muscles. Side effects may include diarrhea, and a fishy odor which may be excreted via the sweat glands. Some patients report decreased fatigue and energy improvements by taking L-Carnitine. It is taken in either tablet or liquid form and is usually taken separate from the compounded cocktail.

### 5) Creatine

Creatine helps maintain muscle mass and increases energy for cells. Its side effects include diarrhea and drowsiness; the dose ranges from 5 grams/day for children to 10 grams/day for adults and is generally compounded into liquid or capsule form.

All of the vitamins and supplements described above are added or not added to a cocktail as specified by a patient's need. Each cocktail is patient specific and can be determined by a physician.

As used herein, the terms “in combination” (as in the sentence “in combination therapy”) or “co-administration” can be used interchangeably to refer to the use of more than one therapy. The use of the terms does not restrict the order in which therapies are administered to a subject.

For combination treatment with more than one therapeutic agents where the therapeutic agents are in separate dosage formulations, or dosage forms, the therapeutic agents may be administered separately or in conjunction (i.e., at the same time). In addition, when administered separately, the administration of one therapeutic agent may be prior to or subsequent to the administration of the other agent.

When Compound I or a pharmaceutically acceptable salt thereof is used in combination therapy with other therapeutic agents, a therapeutically effective amount of the other therapeutic agent or each of the other therapeutic agents will depend on the type of drug used. Suitable dosages are known for approved therapeutic agents and can be adjusted by the skilled artisan according to the condition of the subject, the type of condition(s) being treated and the amount of a Compound I or a pharmaceutically acceptable salt thereof being used. In one embodiment of this invention, Compound I or a pharmaceutically acceptable salt thereof, and the additional therapeutic agent are each administered in an therapeutically effective amount (i.e., each in an amount which would be therapeutically effective if administered alone). In other embodiments, Compound I or a pharmaceutically acceptable salt thereof and the additional therapeutic agent are each administered in an amount which alone does not provide a therapeutic effect (a sub-therapeutic dose). In yet another embodiment, Compound I or a pharmaceutically acceptable salt thereof can be administered in an effective therapeutic amount, while the additional therapeutic agent is administered in a sub-therapeutic dose. In still another embodiment, Compound I or a pharmaceutically acceptable salt thereof can be administered in a sub-therapeutic dose, while the additional therapeutic agent is administered in a therapeutically effective amount.

When co-administration involves the separate administration of a first amount of Compound I or a pharmaceutically acceptable salt thereof and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and pharmacokinetic profile. For example, Compound I or a pharmaceutically acceptable salt thereof and the second therapeutic agent can be administered in any order within 24 hours of each other, within 16 hours of each other, within 8 hours of each other, within 4 hours of each other, within 1 hour of each other, within 30 minutes of each other, within 5 minutes of each other, simultaneously or concomitantly.

More, specifically, a first therapy can be administered prior to (e.g., 5 minutes, 15 minutes, 30 minutes, 45 minutes, 1 hour, 2 hours, 4 hours, 6 hours or 12 hours before)), concomitantly with, or subsequent to (e.g., 5 minutes, 15 minutes, 30 minutes, 45 minutes, 1 hour, 2 hours, 4 hours, 6 hours, 12 hours after), the administration of a second therapy to a subject.

### EXAMPLES

For this invention to be better understood, the following examples are set forth. These examples are for purposes of illustration only and are not be construed as limiting the scope of the invention in any manner. All references provided in the Examples are herein incorporated by reference.

### Example 1: a Signal-Seeking Study to Evaluate Safety, Tolerability, and the Effects of IW-6463 on CNS Disease

Abbreviation/Term Definition

| Abbreviation/Term | Definition |
|---|---|
| AE | adverse event |
| ASL | arterial spin labeling |
| BCRP | Breast cancer resistance protein |
| BOLD | blood-oxygen-level-dependent |
| BP | blood pressure |
| CBF | cerebral blood flow |
| cGMP | cyclic guanosine 3′,5′-monophosphate |
| CNS | central nervous system |
| CRO | clinical research organization |
| CSF | cerebral spinal fluid |
| C-SSRS | Columbia Suicide Severity Rating Scale |
| ECG | electrocardiogram |
| ECHO | echocardiogram |
| EEG | electroencephalogram |
| EOT | end of treatment |
| fMRI | functional magnetic resonance imaging |
| h | hour(s) |
| ICF | informed consent form |
| m | minute(s) |
| MELAS | mitochondrial encephalomyopathy, lactic acidosis, |
| | and stroke-like episodes |
| MFIS | Modified Fatigue Impact Scale |
| MRI | magnetic resonance imaging |
| MRS | magnetic resonance spectroscopy |
| NO | nitric oxide |
| PD | pharmacodynamic(s) |
| PDE5 | phosphodiesterase 5 |
| PGIC | Patient Global Impression of Change |
| PK | pharmacokinetic(s) |
| PROMIS | Patient-Reported Outcomes Measurement |
| | Information System |
| QD | once daily |
| QT | the time between the beginning of |
| | the QRS (ventricular polarization) |
| | complex and the end of the T-wave |
| QTcF | QT interval corrected for HR using |
| | Fridericia's formula |
| SAE | serious adverse event |
| SD | standard deviation |
| sGC | soluble guanylate cyclase |
| SLE | stroke-like episode |
| TEAE | treatment-emergent adverse event |

### Study Objectives and Outcome Measures

This study was completed at 5 investigative sites in the United States. 8 participants completed the study. This study was an open-label, single-arm study evaluating the safety, tolerability, PK, and pharmacodynamics (PD) of Compound I when administered for up to 29 days to participants diagnosed with MELAS. Because this was the first study evaluating Compound I in a patient population, the primary objective was safety and tolerability. The overall study duration for participants was a maximum of 75 days, including the screening, treatment and follow up periods (ClinicalTrials.gov identifier NCT04475549).

A goal of the study described in the Examples section (Example 1) was to assess the effect of a daily dose of 15 mg of Compound I on a number of parameters or measures related to brain and general health, in patients with the mitochondrial disease MELAS. The different assessments and measurements carried out are described in detail below.

The primary objective of the trial was to evaluate the safety and tolerability of Compound I when administered to a patient population of subjects with MELAS, by measuring AEs, SAEs and TEAEs leading to drug discontinuation.

Exploratory objectives include to evaluete the plasma PK of the compund. In addition, other exploratory endpoinds were aimed at evaluating the effect of Compound I on physiology, neurophysiologyand cognitive and health status.

### Study Design:

To complete the study, each participant was to progress through 3 distinct periods as described below. A study schematic is also shown below.

### Screening Phase:

The Screening period began with the execution of the informed consent form (ICF) at the screening visit. After signing the ICF, each participant's study eligibility was assessed according to the Inclusion Criteria and Exclusion Criteria described below. Collection of adverse events (AEs) began after the ICF was signed and continued through the Follow-up period/discontinuation from the study (whichever occured first).

After the Screening visit, participants who were eligible for enrollment had to begin complying with the Lifestyle Restrictions also detailed below. They also began completing their daily diary for the study. The end of the Screening period coincided with the start of the In-Clinic period.

### Treatment Period:

The Treatment Period began on Day 1 (there was no “Day 0”) when participants returned to the Study Center to undergo baseline procedures and receive their first daily dose of study drug. Participants returned to the Study Center on Day 29 (−4 days) for the End-of-Treatment (EOT) Visit; all other scheduled visits during this period were completed within the allowable timeframe either at home or in the Study Center, per participant preference. Throughout the Treatment Period, participants continued completing their daily diary.

The end of the Treatment Period coincided with the beginning of the Follow-up Period.

### Follow-Up Period:

The Follow up period began immediately after the EOT Visit and continued until the Follow-up visit was conducted. During this period, participants had to continue complying with all Lifestyle Restrictions and continued to complete their diary.

### Participant Diary for Recording Changes in Health Status, Daily Dosing, and Concomitant Medications

At the Screening Visit, each participant was issued a paper (source) diary in which they (or their legal representative/guardian) were asked to record on a daily basis the name of any other medication(s) they had taken, along with the date, time, and dose strength of the medication(s); and any changes in their health status (including the dates, times, and brief descriptions).

Starting on Day 1 of the Treatment Period, they were instructed to also record the date and time of each study drug dose taken at home, in addition to any other medications they took (including name, date, time, and dose strength), and any changes in their health status (including the dates, times, and brief descriptions).

Participants were asked to have the diaries available at each scheduled at-home and in-clinic visit during the Treatment Period for monitoring by the study staff.

During the Follow-up Period, participants were asked to continue to record in the same manner any concomitant medications and changes in their health status. The diaries were collected from the participants at the Follow-up Visit.

### Clinical Outcomes Assessments/Participant Questionnaires

### Patient-Reported Outcomes Measurement Information System (PROMIS)-Cognitive Function

The Patient-reported Outcomes Measurement Information System (PROMIS) Item Bank v2.0—Cognitive Function is a self-administered questionnaire that assesses multiple aspects of mental fatigue and cognitive function in the past 7 days prior to the administration of the questionnaire. It uses a Likert-type rating scale (eg, “Never” to “Very often”).

### Patient Global Impression of Change (PGIC)

The Patient Global Impression of Change (PGIC) is a single-item questionnaire that assesses the participant's perception of change in his/her overall health status since the start of the study using a Likert-type rating scale (eg, “Very much improved” to “Very much worse”).

### Modified Fatigue Impact Scale (MFIS)

The Modified Fatigue Impact Scale (MFIS) is a self-administered questionnaire that assesses the impact of fatigue in terms of physical, cognitive, and psychosocial functioning over the past 4 weeks. Patients respond using a Likert-type rating scale. (eg, “Never” to “Almost always”).

### PD Evaluations and Parameters (Neuroimaging)

Two exploratory functional neuroimaging modalities, arterial spin labeling (ASL) and functional magnetic resonance imaging (fMRI), were used to measure the potential PD effects of Compound I in the brain. While ASL quantifies regional CBF during the resting state, fMRI is a relative measure based on the blood-oxygen-level-dependent (BOLD) effect and was conducted during both a resting state and during a visual stimulus to measure brain activity.

Each individual MRI scanning session took approximately 45 minutes (maximum of ˜1 hour).

### Suicidal Risk Monitoring (C-SSRS)

Compound I is a CNS-active investigational drug. Although Compound I and other similar drugs in this class have not been associated with an increased risk of suicidal thinking or behavior, it was considered it important to monitor for such ideation or behavior before and during this clinical study. Therefore, participants were appropriately monitored and closely observed for suicidal ideation and behavior or any other unusual change in behavior. The Columbia Suicidality Severity Rating Scale (C-SSRS) was administered, starting predose on Day 1 of the Treatment Period and at all subsequent visits where clinical assessments were conducted, including at any unscheduled visit. Immediate consultation with the Medical Monitor was to be sought for participants who experienced signs of suicidal ideation or behavior, and consideration given to discontinuing study drug.

### Physical Examination and Vital Signs

At Screening and Follow-up, a complete physical examination was performed by the investigator. All other examinations may be symptom directed at the investigator's discretion.

A complete physical examination was to include examination and assessment of the following: general appearance, lymph nodes, nervous system; cardiovascular system; head, eyes, ears, nose, throat and skin; respiratory system, neck, mental status, abdomen/liver/spleen and musculoskeletal system. Breast, genitourinary, and rectal examinations were optional and were to be performed at the discretion of the investigator. Clinically relevant findings that were present before study drug initiation on Day 1 were recorded as part of the participant's medical history. Beginning after study drug initiation on Day 1, new clinically relevant findings or worsening of an earlier finding were recorded as an AE. Height (cm) was measured only at Screening. Weight (kg) was recorded throughout the study. Body mass index was calculated and recorded at Screening.

Vital signs measured included respiratory rate, oxygen saturation ant temperature. Respiratory rate and oral temperature (° C.) were measured after the participant had been resting supine/seated for ≥5 minutes. Oxygen saturation measurements should be taken by pulse oximeter on room air.

All BP and pulse measurements were obtained with an automated BP device (left arm was preferred) prior to blood draws (or ≥10 m after a blood draw, if necessary). Supine BP was taken at Screening (only) and recorded as the average of 2 measurements obtained at 2-minute intervals after the participant had rested quietly in a semi-recumbent/supine position for ≥5 minutes. Orthostatic vital signs were taken at all scheduled visits and participant was to rest quietly in a supine/semi-recumbent position for ≥5 minutes before supine BP and pulse measurements were recorded, then assume sitting position for ≥1 minute, and finally assume a standing position for 2 (±1) minutes before standing measurements were recorded. Values from these measurements were used to calculate and record orthostatic BP and pulse.

### Electrocardiograms (ECGs)

All ECGs and the Screening ECHO (if not documented in medical history within 3 months prior to Day 1) were obtained after the participant had rested supine for ≥5 minutes and were obtained before blood draws (or ≥10 m after a blood draw, if necessary). When timing coincided, ECGs and vital signs could be assessed together.

If a QTc result (corrected using Fridericia's formula) was outside of the normal range (>450 ms), the ECG was to be repeated twice and the average of the 3 results calculated.

All ECGs were evaluated by an Investigator or qualified designee for the presence of abnormalities. Results were reported as “normal,” “abnormal clinically significant,” or “abnormal not clinically significant.” Abnormalities of clinical significance were recorded as an AE.

If a confirmed clinically significant abnormal result was obtained, the Study Center was to follow standard institutional procedure until the result resolves to baseline. If concerns remained, the issue was escalated to the Sponsor's Medical Monitor.

### Adverse Events, SAEs, and Other Safety Reporting

Adverse events (AEs) were monitored and recorded throughout the study starting from ICF execution through the Follow-up Visit. Care was taken not to introduce bias when detecting AEs and/or SAEs. Open-ended and nonleading verbal questioning of the participant were preferred method to inquire about AE occurrences.

Investigators were not obligated to actively collect AE information after conclusion of the participant's involvement in the study. However, if the Investigator learned of any SAE or death at any time after a participant has been discharged and the Investigator considered the event to be reasonably related to the study drug or to study participation, he/she was to promptly notify the Sponsor.

### Pregnancy Reporting and Monitoring

A female participant who reported a pregnancy before initiation of study drug dosing on Day 1 was to be withdrawn from study participation; the discontinuation was to be recorded as a screen failure.

If a pregnancy occured in either a participant or a participant's partner after initiation of study drug, it was reported and recorded on the study-specific pregnancy form. The Study Center should was to make reasonable efforts to follow the pregnancy to term.

All reports of congenital abnormalities/birth defects and still births were to be considered SAEs. Spontaneous miscarriages, elective termination without complications, and normal births without congenital abnormality were not to be reported or handled as SAEs, but reported as the outcome of the respective pregnancy.

### Other Assessments

A variety of clinical laboratory assessments for safety, drug testing, and otherwise required by protocol were carried out in all subjects. Genetic evaluation in this study was limited to confirmation of MELAS (by medical history) at Screening for eligibility determination.

### Plasma and CSF PK and Biomarker Evaluations

PK samples were collected. Each sample was divided into 2 aliquots (1 each for PK and a back-up). Sparse whole-blood samples of approximately 2 mL were collected for measurement of plasma concentrations of Compound I using a validated liquid chromatography-tandem mass spectrometry bioanalytical method.

From each participant who did not withhold consent for the procedure, a CSF sample of approximately 4 mL was collected for measurement of Compound I CSF concentration using a validated liquid chromatography-tandem mass spectrometry bioanalytical method.

Plasma and serum samples for biomarkers were collected from each participant. These biomarkers tested the target engagement of Compound I as well as the impact of the compound on disease. Plasma or serum samples were analyzed for concentrations of growth differentiation factor 15 (GDF-15), lactate, pyruvate, alanine, neurofilament light chain (NFL), vascular cell adhesion molecule 1, interleukin-1beta, asymmetric dimethylarginine (ADMA), L arginine, and other biomarkers relevant to the pathophysiology of mitochondrial disease, as well as their response to Compound I.

The Compound I effect on the neuronal profile of the brain was also measured in participants by proton magnetic resonance spectroscopy (¹H-MRS). Biomarkers measured using this imaging technology included ventricular lactate and N-acetylaspartate concentrations amongst others.

### Study Drug, Dose Levels and Administration

All eligible participants received open-label Compound I at a starting dose of 15 mg QD. Participants were instructed to take Compound I once per day on Days 2 through 28 at a time consistent (preferably ±1 hour) with the in-clinic study drug administration on Day 1.

Exception/Note: For the Day 8, 15, and 29/EOT visits, participants took their dose during the visit to allow for the appropriate timing of pre- and postdose assessments. Day 8 and 15 visits (at home or in clinic) were scheduled such that dosing could still occur at approximately the same time as the Day 1 and subsequent doses. Participants were to record the date and time of each at-home dose administration in their diary.

Permitted concomitant medications could be taken with study drug. Participants were asked to record in their diary each dosing day, time, and dose strength of any concomitant medication(s) they took.

While Compound I could be taken with or without food, participants were requested to fast for 3 to 4 hours prior to in-clinic and at-home clinical safety and PD laboratory sample collections.

The 15-mg Compound I QD dose was selected based on safety, tolerability, PK, and PD data from repeated dosing at this level in two Phase 1 studies conducted in healthy adults aged 18 to 79 years. Among the healthy participants who received 15 mg Compound I QD for up to 15 days, there were no discontinuations due to AEs and no SAEs reported. All AEs were considered mild or moderate by the investigator. Across the Phase 1 studies, no safety concerns were identified. PK data were linear and dose-dependent, were supportive of QD dosing, and were not impacted by food. Additionally, after 14 days of dosing in healthy elderly participants, 15 mg Compound I QD demonstrated modest impacts on neurophysiological parameters by EEG. No impact on cognitive performance measures was observed.

### Participant Selection and Lifestyle Restrictions

### Inclusion Criteria

Each participant had to meet each of the following criteria to be eligible for entering this study:
1. Had signed an ICF (either the participant or his/her legal representative/guardian, where appropriate) before any study-specific procedures are performed
2. Was 18 years of age or older on the day of consent
3. Had received prior genetic confirmation of a known mitochondrial disease mutation
4. Displayed neurological features of MELAS (could be based on medical history)
5. Had elevated plasma lactate during Screening (≥1.0 mmol/L)
6. Agreed to adhere to all study requirements, including Lifestyle Restrictions
7. Agreed to refrain from making any major lifestyle changes (eg, start a new diet or change an exercise pattern) from the time of the ICF signature until after the Follow-up Visit (and beyond, vis-à-vis the contraception requirements)
8. If receiving chronic medication(s), had no change in regimen(s) for ≥4 weeks before first day of dosing and had no plans to change regimen(s) during the study
9. If female, met 1 of the 2 following criteria: Confirmed as being postmenopausal (no menses for ≥1 year or ≥12 consecutive months) or surgically sterile (bilateral oophorectomy, hysterectomy, or tubal sterilization [tie, clip, band, or burn]) or if of reproductive potential was not pregnant or breastfeeding at the time of the Screening Visit and had negative pregnancy test results at the Screening Visit and predose at Day 1 and agreed to contraception for duration of study and for ≥90 days after the final study drug dose
10. Male and female participants of reproductive potential had to agree to use ≥1 of a series of effective contraception methods from the date of signing the ICF until ≥90 days after receiving his/her final study drug dose.
11. Male participants had to agree to refrain from donating sperm from the Screening Visit through 90 days after their final dose of study drug.
12. Agreed to not receive an investigational therapy or device in any other study while participating in this study, through the Follow-up Visit
13. Had not had clinically significant findings on ECG (as assessed at Screening and predose on Day 1) and echocardiogram (ECHO; within 3 months of Day 1)

### Exclusion Criteria

A participant who met any of the following criteria was excluded from entering this study.
1. Displayed severe visual, auditory, or cognitive impairment as determined by the Investigator that may affect the ability to adhere to protocol requirements or to complete required assessments
2. Used any nicotine-containing products (eg, cigarettes, e-cigarettes, vape pens, cigars, chewing tobacco, gum, patches) within 1 month of enrollment
3. Had a positive pregnancy test at Screening or on Day 1 (or at any other point during the study)
4. Had received inpatient hospitalization for alcoholism or drug addiction in the 12 months before the Screening Visit and/or had positive drug or alcohol test results at Screening or predose at Day 1. Drug screen included amphetamines, cocaine, opiates, and cannabinoids. Noate that use of cannabis and cannabidiol products for medical purposes was permitted in this study except for 24 hours before the Screening, Day 1, and EOT visits, and for 4 hours prior to all other visits. A participant was excluded from study entry if there was a known cannabis abuse or dependence that, in the opinion of the Investigator, impacted the ability of that individual to comply with the protocol or may lead to harm to the individual.
5. Showed clinically significant hypersensitivity or allergy to any of the inactive ingredients contained in the Compound I drug product
6. Displayed hypotension, defined as systolic BP ≤90 mmHg or diastolic BP ≤60 mmHg at Screening or predose at Day 1
7. Displayed hypertension, defined as systolic BP >160 mmHg or diastolic BP >100 mmHg, at Screening or predose at Day 1
8. Displayed orthostatic hypotension at Screening or predose at Day 1, defined as a decrease in systolic BP of ≥20 mmHg or a decrease in diastolic BP of ≥10 mmHg when measured after assuming a standing position from a semi-recumbent/supine position
9. Had uncontrolled diabetes mellitus with an HbAlc >11% or as determined by the Investigator
10. Had had lymphoma, leukemia, or any malignancy within the past 5 years with the exception of basal cell or squamous epithelial carcinomas of the skin that had been resected with no evidence of metastatic disease for 3 years.
11. Had severe gastrointestinal dysmotility (eg, history of dyspepsia, stomach aches, nausea, vomiting, recurrent pancreatitis, constipation) as determined by the Investigator that may impact compliance and/or oral drug administration, absorption, and exposure
12. Was unable to fast (ie, no food or fluid; water allowed as required) for 3 to 4 hours after a meal
13. Had a family history of short QT syndrome or long QT syndrome
14. Had clinically significant cardiac involvement or an ECG with corrected QT interval using Fridericia's formula (QTcF interval) >500 ms
15. Had a current or past history of clinically significant cardiomyopathy and/or cardiac conduction abnormality
16. Had a history of platelet dysfunction, hemophilia, von Willebrand disease, coagulation disorder, other bleeding diathesis condition(s), or significant, nontraumatic bleeding episodes
17. Was using aspirin ≥325 mg/day, any P2Y12 inhibitor, any anticoagulant medication, specific inhibitors of phosphodiesterase 5 (PDE5), nonspecific inhibitors of PDE5 (including dipyridamole and theophylline), any supplement for the treatment of erectile dysfunction, riociguat, and/or any nitrate. These medications were prohibited from the Screening Visit through the duration of the study. Note: Participants who were taking arginine or citrulline for the treatment of mitochondrial disease were eligible and allowed to continue these therapies.
18. Participated in any study of investigational therapies for mitochondrial disease and/or for the symptoms of mitochondrial disease within 1 month before Day 1
19. Had any contraindication to MRI procedures
20. Had been hospitalized for any illness, trauma, surgical procedure, or mitochondrial disease-related complication within 4 weeks before Screening
21. Was unable or unwilling to adhere to the study schedule, lifestyle restrictions and assessment requirements or, in the clinical judgment of the Investigator, was otherwise not suitable for study participation
22. Patient or his/her legal representative/guardian (where appropriate) were unable or unwilling to provide written, informed consent to participate in this study.

### Lifestyle Restrictions

Participants were to abide by the following lifestyle restrictions throughout the study, starting from the Screening Visit through the Follow-up Visit, unless otherwise indicated.

### Meals and Dietary Restrictions

Fasting (ie, no food or fluid; water allowed as required) was required for 3 to 4 hours prior to the collection of clinical safety and PD laboratory samples at each scheduled visit, including at any at-home visit.

Because participants could be vulnerable to metabolic decompensation during any catabolic state, on the day of each visit, participants fasted upon awakening (water and a light snack, if previously approved, are allowed) and then were given a standardized meal with a low glycemic index in the clinic, 3 to 4 hours before the collection of clinical safety and PD laboratory samples. For at-home visits, participants were either counseled on what to consume prior to the fasting period or were given a standardized meal by home health services.

On days when neuroimaging was conducted (Screening, Day 1, and Day 29 visits): After the safety and PD laboratory samples had been collected, participants were allowed to eat a light standardized snack; heavy meals, however, had to be avoided until after all imaging scans had been conducted.

### Caffeine, Alcohol, and Tobacco

Caffeine- or xanthine-containing products (eg, coffee, tea, cola drinks, and chocolate) could not be ingested for 24 hours before the Screening, Day 1, and EOT visits, and for 4 hours prior to all other scheduled visits. These products can impact the assessments conducted in this study.

The use of alcohol was not permitted during the 24 hours prior to any scheduled visit. Tobacco- or nicotine-containing products (eg, e-cigarettes, patches) could not be used during this study until after the final study visit.

### Medications, Vitamins, Supplements, and Other Substances

Participants were to be advised that the following were prohibited as indicated: any medical treatment for erectile dysfunction beginning with Screening through the Follow-up Visit; “illicit” drugs beginning 1 month before Screening through the Follow-up Visit; use of cannabis and cannabidiol products for medical purposes was permitted except for the 24-hour period before Screening, the Day 1 and EOT visits, and for 4 hours before all other visits; foods containing poppy seeds were to be avoided throughout the study because they can cause a positive “drug” result.

### Concomitant Medications and Procedures

Any medication, including over-the-counter or prescriptions medicines, vitamines, and/or herbal supplements, vaccine, or other therapeutic/procedure that the participant was receiving at the time of Screening until the Follow-up Visit was recorded along with the reasons for use, dates of administration, route of administration, and frequency, as well as dosage information. The following medications were prohibited or allowed with caution: specific inhibitors of PDE5, including sildenafil, tadalafil, vardenafil; nonspecific inhibitors of PDE5, including dipyridamole, theophylline; any supplement for the treatment of erectile dysfunction, other sGC stimulators, including riociguat and vericiguat; nitrates such as nitroglycerin, isosorbide mononitrate, isosorbide dinitrate, sodium nitroprusside, amyl nitrate; aspirin ≥325 mg/day; any anticoagulant medication; any P2Y12 inhibitor, including cangrelor, clopidogrel, prasugrel, ticagrelor, ticlopidine; use of any “illicit drug” was not permitted beginning 1 month before Screening through the Follow up Visit, except that use of cannabis and cannabidiol products for medical purposes was permitted, except for 24 hours before the Screening, Day 1, and EOT visits, and for 4 hours prior to all other study visits; and BCRP substrates because Compound I may have the potential to increase the exposures of these medications; inhibitors of BCRP transporters, including curcumin, cyclosporine A and eltrombopag. Examples of BCRP substrates include azidothymidine, bisantrene, camptothecin derivates, canertinib, cimetidine, diflomotecan, flavopiridol, gefitinib, glyburide, imatinib mesylate, indolocarbazole, irinotecan, lamivudine, lapatinib, methotrexate, mitoxantrone, nilotinib, nitrofurantoin, pantoprazole, prazosin, rosuvastatin, SN 38, sulfasalazine, and topotecan.

Participants taking arginine or citrulline for the treatment of mitochondrial disease were allowed to continue these therapies.

### Baseline Characteristics

Of the 8 patients that completed the trial, 5 were taking arginine and one was taking citrulline before and during the trial. Use of arginine or citrulline was more common in patients with more advanced disease.

Patients enrolled spanned between 19 and 54 years old. 5 of them were women and 3 were men. All of them had a history of 1 or more CNS symptoms, such as stroke-like episodes, seizuers or headaches. MFIS scores at baseline spanned between 3 and 66 (on a scale of 0-84). And PROMIS scores spanned between 148 and 77 on a scale of 160 to 0. These patients had between 6 and 19 elevated inflammatory biomarkers at baseline.

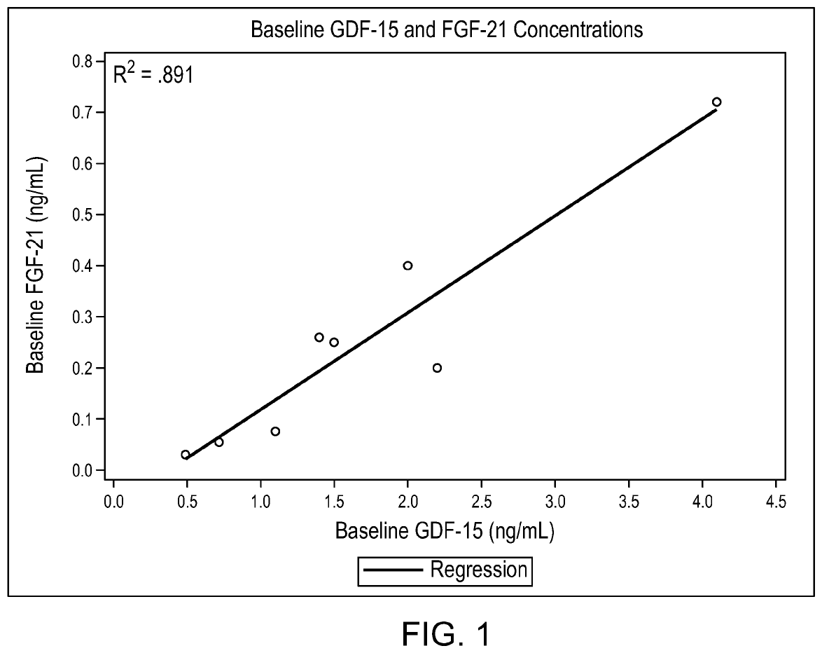

In addition, a correlation of biomarkers of mitochondrial dysfunction GDF-15 and FGF-21 was observed at baseline (see FIG. 1), which further supports evidence of disease distribution amongst the 8 patients.

Thus, even though the trial was completed with a small number of patients it showed a distribution of patients from those that could be considered milder in their disease presentation to those that could be considered to have a more severe mitochondrial disease phenotype.

The levels of plasma lactate at baseline ranged from 1.7 to 5.6 mmol/L. Normal levels of lactate are lower than 2 mmol/L. The level of GDF-15 ranged at baseline between 0.49 and 4.1 ng/mL. Normal levels of this biomarker are between 0.14 and 0.46 ng/mL. The level of FGF-21 at baseline ranged between 0.055 and 0.72 ng/mL. The normal range for FGF is less than 0.44 ng/mL. Therefore, plasma biomarkers linked to mitochondrial dysfunction were elevated at baseline across al participants.

### Study Assessments/Results

### a) Safety

Compound 115 mg QD for 29 days was well tolerated with no SAEs observed and no adverse events leading to treatment discontinuation in patients with the mitochondrial disase MELAS. No safety signals on clinical labs, vital signs, ECGs, or suicidal rating scale were obtained.

### b) Pharmacokinetics

The PK profile and CNS penetrance of Compound I in this patient population was similar to that observed in prior Phase 1 studies.

### c) Pharmacodynamics

### i) Neuroimaging

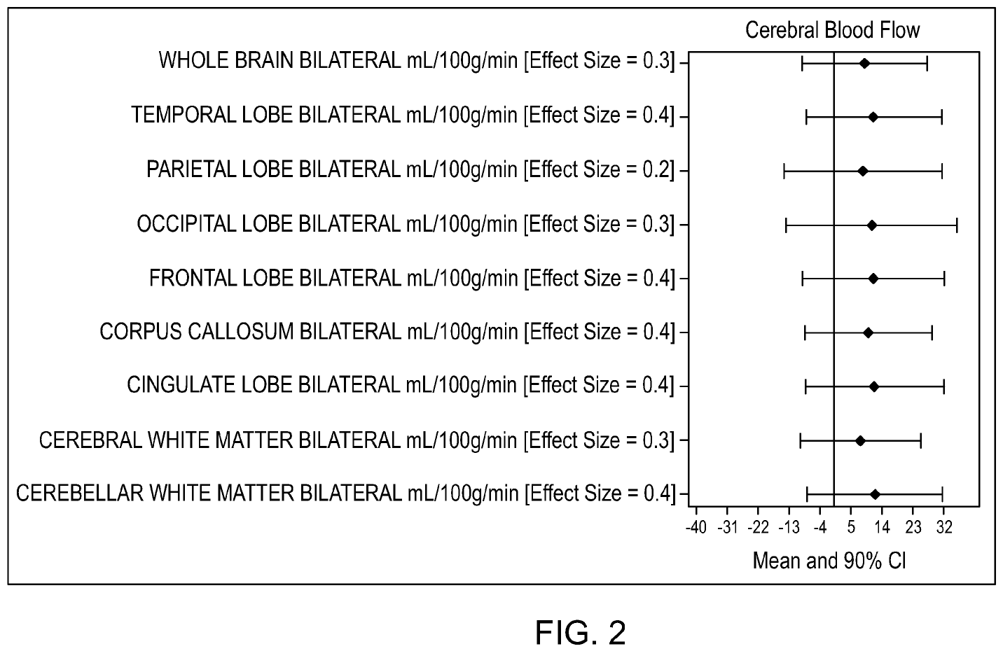

A positive trend across the study was observed as an increase of CBF on day 29 when compared to Baseline (day 1) as shown in FIG. 2. This change was observed across all brain regions of interest. Effect size throughout this disclosure is defined as the means change from baseline in a particular parameter that is being measured, divided by the standard deviation. Effect sizes closer to 1 or −1 (positive or negative dependent on the direction of movement for the specific parameter, i.e. +0.8 to +1.0 or −0.8 to −1.0) are usually considered large. Effect sizes between 0.5 and 0.8 or −0.5 and −0.8 (positive or negative dependent on the direction of movement for the specific parameter) are usually considered moderate. Effect sizes closer to below 0.5 or −0.5 (positive or negative dependent on the direction of movement for the specific parameter) are usually considered small.

When analyzing the data of individual patients, CBF increased in 5 of the 8 patients between day 1 and day 29, with patients having lower CBF at baseline, driving the overall changes.

Task-free functional MRI during resting state showed enhanced connecitivity on Day 29 compared to screening and day 1. Increased signals across several resting state networks were observed including those involved in executive function and sensorimotor processing. Task-based functional MRI (visual activation with a flashing checkerboard pattern) showed increased activation of voxels in the occipital region with Compound I on Day 29 compared to screening and Day 1 visits.

### ii) Biomarkers

26 out of the 40 (65%) inflammatory biomarkers assessed in plasma as part of the InflammationMAP® panel were reduced with effect sizes less or equal than −0.3. Only 3 of the 40 (7.5%) inflammatory biomarkers assessed in plasma increased with an effect size equal or more than 0.3. The changes in concentrations as well as the effect sizes for inflammatory markers measured in plasma are summarized in the 3 tables below (Table 1: large effect size; Table 2: moderate effect size and Table 3: small effect size). The concentration values in Tables 1-3 were measured in the standard units for each of these laboratory measures as known in the art. Many of these biomarkers tended to increase again back to baseline levels after treatment with Compound I was stopped, supporting a drug effect.

TABLE 1
(changes in biomarker concentrations,
biomarkers with large effect size)

| Biomarkers (Large | | | |
|---|---|---|---|
| Effect Sizes) | Mean | 90% CI | Effect size |

| | | | | |
|---|---|---|---|---|
| Beta-2-Microglobulin | −0.5 | −0.7 | −0.2 | −1.0 |
| Serum Amyloid | −5.0 | −8.8 | −1.2 | −0.9 |
| P-Component | | | | |

TABLE 2
(changes in biomarker concentrations,
biomarkers with moderate effect size)

| Biomarkers (Moderate | | | | Effect |
|---|---|---|---|---|
| Effect Sizes) | Mean | 90% CI | | size |
| Tumor necrosis factor receptor 2 | −1.8 | −3.5 | −0.1 | −0.7 |
| Thyroxine-Binding Globulin | −8.4 | −16.4 | −0.4 | −0.7 |
| Ferritin | −21.4 | −43.8 | 1.1 | −0.6 |
| Immunoglobulin M | −0.4 | −0.9 | 0.0 | −0.6 |
| Vascular Cell Adhesion Molecule-1 | −98.3 | −208.7 | 12.2 | −0.6 |
| C-Reactive Protein | −3.4 | −7.6 | 0.7 | −0.6 |
| Brain-Derived Neurotrophic Factor | −2.1 | −4.6 | 0.5 | −0.5 |
| Pulmonary and Activation-Regulated | −15.3 | −34.8 | 4.3 | −0.5 |
| Chemokine | | | | |
| Vitamin D-Binding Protein | −26.5 | −63.0 | 10.0 | −0.5 |
| Immunoglobulin A | −0.4 | −0.9 | 0.2 | −0.5 |
| T-Cell-Specific Protein RANTES | −7.2 | −17.9 | 3.4 | −0.5 |
| Interleukin-17 (IL-17) | 0.03 | −0.01 | 0.06 | 0.5 |

TABLE 3
(changes in biomarker concentrations,
biomarkers with small effect size)

| Biomarkers (Small | | | | Effect |
|---|---|---|---|---|
| Effect Sizes) | Mean | 90% CI | | size |
| EN-RAGE | −28.4 | −70.7 | 14.0 | −0.4 |
| Myoglobin | −32.5 | −81.4 | 16.3 | −0.4 |
| Fibrinogen | −1.4 | −3.5 | 0.8 | −0.4 |
| Interleukin-18 | −25.6 | −65.5 | 14.2 | −0.4 |
| Plasminogen Activator Inhibitor 1 | −11.6 | −29.9 | 6.7 | −0.4 |
| von Willebrand Factor | −123.4 | −321.2 | 74.4 | −0.4 |
| Factor VII | −87.4 | −242.3 | 67.5 | −0.4 |
| Alpha-1-Antitrypsin | −0.2 | −0.6 | 0.2 | −0.4 |
| Eotaxin-1 | −10.1 | −29.0 | 8.8 | −0.4 |
| Granulocyte-Macrophage Colony- | −13.6 | −39.4 | 12.2 | −0.4 |
| Stimulating Factor | | | | |
| Alpha-2-Macroglobulin | −0.2 | −0.6 | 0.2 | −0.3 |
| Intercellular Adhesion Molecule 1 | −12.1 | −41.9 | 17.6 | −0.3 |
| Vascular Endothelial Growth Factor | −7.0 | −25.1 | 11.1 | −0.3 |
| Interleukin-10 (IL-10) | 0.3 | −0.2 | 0.7 | 0.4 |
| Matrix Metalloproteinase-9 (MMP-9) | 37.3 | −18.8 | 93.3 | 0.4 |

Beta-2-microglobulin (B2M) is a circulating factor that negatively regulates cognitive regenerative function in the adult hippocampus in an age-dependent manner.

Compound I improved biomarkers with known associations to MELAS. Expression of VCAM-1 (vascular cell adhesion molecule-1), ICAM (intercellular adhesion molecule) and vWF (von Willebrand factor) is higher in endothelial cells from patients with MELAS. VCAM and ICAM are endothelial adhesion molecules that can promote pathological plaque formation and that may constribute to stroke-like-episodes. vWF is a marker of endothelial activation and damage, which is elevated in patients with clear vasculopathy. Expression of TNFR2 (tumor necrosis factor receptor) is higher in muscle biopsies from patients with mitochondrial respiratory chain dysfunction. The biological effects of TNF-alpha in mitochondrial diseases are mainly mediated by TNFR2.

In this trial, changes in CBF were strongly correlated with changes in B2M as seen in Table 4 below (correlations of higher or equal than 0.8 are considered very strong; correlations of between or equal to 0.6 and 0.8 are considered strong).

TABLE 4
Correlations between changes in ASL
(CBF) increases and B2M decreases

| ASL parameters | |
|---|---|
| (mL/100 g/min) | Beta2 microglobulin |
| CEREBELLAR WHITE MATTER | −0.93 |
| CEREBRAL WHITE MATTER | −0.68 |
| CINGULATE LOBE | −0.61 |
| CORPUS CALLOSUM | −0.53 |
| FRONTAL LOBE | −0.65 |
| OCCIPITAL LOBE | −0.77 |
| PARIETAL LOBE | −0.71 |
| TEMPORAL LOBE | −0.65 |
| WHOLE BRAIN | −0.73 |

Serum amyloid P-component is a small glycoprotein found in normal serum and in all amyloid deposits. It acts as an acute phase protein, modulates immunologic responses, inhibits elastase and has been suggested as an indicator of liver disease or neurological disorders.

Tumor necrosis factor receptor 2 (TNFR2) is expressed in muscle fibers with abnormal focal accumulations of mitochondria and is delivered to mitochondria where receptor is localized.

Several biomarkers of mitochondrial dysfunction were also measured in plasma. Lactate concentrations, which are known to be elevated in mitochondrial disease patients, showed a trend towards reduction with an effect size of −0.5. GDF-15, a biomarker of mitochondrial disease was also measured in plasma and showed a trend towards reduction with an effect size of −0.5. FGF-21, another biomarker of mitochondrial disease was also measured in plasma and showed a trend towards reduction with a smaller effect size of −0.2. There were directional improvements with treatment and changes in biomarkers of mitochondrial dysfunction were correlated with trough Compound I plasma concentrations on day 29 as can be seen in Table 5 below. That is, higher trough concentrations of Compound I at day 29 were associated with larger decreases in of mitochondrial dysfunctionas seen in Table 5 below (correlations of higher or equal than 0.8 are considered very strong; correlations of between or equal to 0.6 and 0.8 are considered strong). In addition, strong correlations were observed between changes in biomarkers of mitochondrial dysfunction GDF-15, FGF-21 and lactate as well as serum amyloid P component at day 29.

Reductions in lactate were observed in 6 of 8 participants and ranged from 7% to 46%. Reductions in GDF-15 were observed in 4 of 8 participants with greatest reductions (up to 39%) in participants with higher baseline concentrations. Changes in these biomarkers of mitochondrial dysfunction were strongly correlated with each other and also correlated with CY6463 plasma concentrations at the end of treatment.

TABLE 5
Correlations between biomarkers of mitochondrial dysfunction,
inflammatory biomarkers and Compound I concentration

| Biomarker | | | | | | | | | Trough plasma |
|---|---|---|---|---|---|---|---|---|---|
| parameters | FGF-21 | GDF-15 | SAPC | Lactate | VCAM-1 | ICAM-1 | TNFR2 | vWF | concentration |
| Fibroblast growth | 1.00 | | | | | | | | |
| factor 21 | | | | | | | | | |
| Growth | 0.86 | 1.00 | | | | | | | |
| differentiation | | | | | | | | | |
| factor 15 | | | | | | | | | |
| VCAM-1 | 0.58 | 0.40 | | 0.15 | 1.00 | | | | |
| ICAM-1 | 0.86 | 0.89 | | 0.74 | 0.56 | 1.00 | | | |
| TNFR2 | 0.40 | 0.42 | | 0.15 | 0.89 | 0.56 | 1.00 | | |
| vWF | 0.85 | 0.92 | | 0.83 | 0.36 | 0.71 | 0.31 | 1.00 | |
| Serum amyloid P | 0.85 | 0.72 | 1.00 | | | | | | |
| component | | | | | | | | | |
| Lactate | 0.74 | 0.87 | 0.60 | 1.00 | | | | | |
| Trough plasma | −0.75 | −0.68 | −0.64 | −0.41 | −0.54 | −0.76 | −0.40 | −0.66 | 1.00 |
| concentration | | | | | | | | | |
| Compund I | | | | | | | | | |

correlations of ≥0.8, very strong; correlations ≥0.6 but <0.8, strong; correlations ≥0.4 but <0.6, moderate
FGF = fibroblast growth factor;
GDF = growth/differentiation factor;
ICAM = intercellular adhesion molecule;
TNFR2 = tumor necrosis factor receptor 2;
VCAM = vascular cell adhesion molecule;
vWF = von Willebrand factor

iii) Subjective Assessments PROs

Three of the 8 participants enrolled reported improvement in their disease (1 participant minimally improved, 1 participant much improved, and 1 participant very much improved), 4 participants reported no change in their disease and 1 participant reported their disease as much worse. Even though changes at the study level were not observed in the MFIS and PROMIS assessment tools, some relevant observations can be made at the patient level. Participants who responded more positively on these PROs at baseline and with milder disease did not show improvements on Day 29, while participants who responded more poorly on these assessments and with more advanced disease at baseline reported more improvement.

As seen in Table 6 below, interesting corrections were also observed between CBF and clinical improvement as assessed by the patient global impression of change change (PGIC). Correlations equal or above 0.8 are considered very strong, and between 0.6 and 0.8 are strong.

TABLE 6
Correlations between CBF and clinical improment as measured by PGIC

| | Cerebellar | Cerebral | | | | | | | |
|---|---|---|---|---|---|---|---|---|---|
| ASL | white | white | Cingulate | Corpus | Frontal | occipital | Parietal | Temporal | Whole |
| parameters | matter | mater | lobe | callosum | lobe | lobe | lobe | lobe | brain |
| PGIC | −0.65 | −0.85 | −0.90 | −0.78 | −0.82 | −0.75 | −0.79 | −0.85 | −0.84 |

1. A method of treating mitochondrial disease in a patient in need thereof comprising administering to said patient a total oral daily dose of between 15 mg and 60 mg of Compound I:

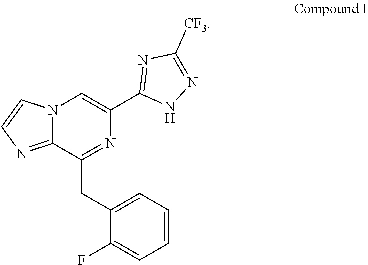 [SMILES: Fc1ccccc1Cc1nc(-c2nc(C(F)(F)F)n[nH]2)cn2ccnc12]

2. The method of claim 1, wherein the mitochondrial disease is selected from Alpers Disease, Autosomal Dominant Optic Atrophy (ADOA), Barth Syndrome/LIC (Lethal Infantile Cardiomyopathy), Beta-oxidation defects, Long Chain Fatty Acid Transport Deficiency, Co-Enzyme Q10 Deficiency, Complex I, II, III, IV, V Deficiency, Chronic Progressive External Ophthalmoplegia (CPEO), Friedreich's Ataxia, Kearns-Sayre syndrome, Leukodystrophy, Leigh Disease or Syndrome, LHON, LHON Plus, MELAS (Mitochondrial myopathy, encephalomyopathy, lactic acidosis, stroke-like symptoms), Myoclonic Epilepsy with Ragged Red Fibers (MERRF), Mitochondrial Recessive Ataxia Syndrome (MIRAS), Mitochondrial Cytopathy, Mitochondrial DNA Depletion, Mitochondrial Encephalopathy, Mitochondrial Myopathy, Multiple Mitochondrial Dysfunction Syndrome, MNGIE (Myoneurogenic gastrointestinal encephalopathy), NARP (Neuropathy, ataxia, retinitis pigmentosa, and ptosis), Pearson Syndrome, Pyruvate Carboxylase Deficiency, Pyruvate Dehydrogenase Deficiency or Pyruvate Dehydrogenase Complex Deficiency (PDCD/PDH), and POLG Mutations.

3. The method of claim 1, wherein the mitochondrial disease is selected from Alpers, Complex I, II, III, IV deficiency, CPEO, KSS, LCHAD, Leigh syndrome, Leukodystrophy, LHON, MELAS, MEPAN, MERRF, MIRAS, Mitochondrial DNA depletion, MNGIE, NARP, Pearson syndrome, and POLG mutations.

4. The method of claim 1, wherein the mitochondrial disease is a Complex I mitochondrial disease.

5. The method of claim 1, wherein the mitochondrial disease is MELAS.

6. The method of claim 1, wherein the mitochondrial disease is Leigh syndrome.

7. The method of any one of claims 1-6, wherein the patient is 16 years or older, 18 years or older or 65 years or older.

8. The method of any one of claims 1-6, wherein the patient is between 16 and 75, between 16 and 70, between 16 and 65, between 16 and 60, between 16 and 55, between 16 and 50, between 16 and 40, or between 16 and 30 years old.

9. The method of any one of claims 1-6, wherein the patient is younger than 65 years old, younger than 60 years old, younger than 50 years old, younger than 40 years old, younger than 30 years old or younger than 20 years old.

10. The method of any one of claims 1-6, wherein the patient is 16 years old or younger, 12 years old or younger, 10 years old or younger, or 5 years old or younger.

11. The method of any one of claims 1-6, wherein the patient is between 3 and 18, between 3 and 12, between 5 and 18, between 5 and 12, or between 3 and 5 years old.

12. The method of any one of claims 1 to 11, wherein the patient is administered a total oral daily dose of 15 mg of Compound I.

13. The method of claim 12, wherein the patient is administered a single oral daily dose of 15 mg of Compound I.

14. The method of claim 12, wherein the patient is administered two oral daily doses of 7.5 mg of Compound I.

15. The method of any one of claims 1 to 11, wherein the patient is administered a total oral daily dose of 20 mg of Compound I.

16. The method of claim 15, wherein the patient is administered a single oral daily dose of 20 mg of Compound I.

17. The method of claim 15, wherein the patient is administered two oral daily doses of 10 mg of Compound I.

18. The method of any one of claims 1 to 11, wherein the patient is administered a total oral daily dose of 25 mg of Compound I.

19. The method of claim 18, wherein the patient is administered a single oral daily dose of 25 mg of Compound I.

20. The method of claim 18, wherein the patient is administered two oral daily dose of 12.5 mg of Compound I.

21. The method of any one of claims 1 to 11, wherein the patient is administered a total oral daily dose of 30 mg of Compound I.

22. The method of claim 21, wherein the patient is administered a single oral daily dose of 30 mg of Compound I.

23. The method of claim 21, wherein the patient is administered two oral daily dose of 15 mg of Compound I.

24. The method of any one of claims 1 to 11, wherein the patient is administered a total oral daily dose of 45 mg of Compound I.

25. The method of claim 24, wherein the patient is administered a single oral daily dose of 45 mg of Compound I.

26. The method of claim 24, wherein the patient is administered two oral daily dose of 22.5 mg of Compound I.

27. The method of any one of claims 1 to 11, wherein the patient is administered a total oral daily dose of 60 mg of Compound I.

28. The method of claim 27, wherein the patient is administered a single oral daily dose of 60 mg of Compound I.

29. The method of claim 24, wherein the patient is administered two oral daily dose of 30 mg of Compound I.

30. The method of claim 14, 17, 20, 23, 26 or 29, wherein the patient is administered a first dose and a second dose, wherein the first dose and the second dose are separated by a period between 5 hours and 15 hours, between 8 hours and 15 hours, or between 10 hour and 15 hours.

31. The method of any one of claims 1 to 30, wherein treatment with Compound I results in a measurable improvement in neuronal function and connectivity.

32. The method of claim 31, wherein the improvement in neuronal function and connectivity is measured by functional magenic resonance imaging (fMRI).

33. The method of any one of claims 1 to 32, wherein treatment with Compound I results in an increase in cerebral blood flow (CBF).

34. The method of claim 33, wherein the treatment with Compound I results in an increase in cerebral blood flow (CBF) in a brain region selected from temporal lobe, parietal lobe, occipital lobe, frontal lobe, corpus callosum, cingulate lobe, cerebral white matter and cerebellar white matter, or a combination of one or more aforementioned regions.

35. The method of any one of claims 1 to 34, wherein treatment with Compound I results in a reduction in one or more biomarkers of mitochondrial dysfunction.

36. The method of claim 35, wherein the one or more biomarkers of mitochondrial dysfunction are selected from lactate, GDF-15 and FGF-21.

37. The method of any one of claims 1 to 36, wherein treatment with Compound I results in a reduction in one or more inflammatory biomarkers.

38. The method of claim 37, wherein the one or more inflammatory biomarkers are selected from VCAM-1, ICAM, vWF, and TNFR2.

39. The method of any one of claims 1 to 38, further comprising administering to the patient one or more additional therapeutic agent.

40. The method of claim 39, wherein the additional therapeutic agent is selected from citrulline and arginine.

41. The method of claim 39, wherein the additional therapeutic agent is a mito cocktail.

42. The method of any one of claims 1 to 41, wherein the patient has been treated with one or more other therapeutic agent used for treating mitochondrial disease.

43. The method of claim 42, wherein the other therapeutic agent used for treating mitochondrial disease is selected from citrulline and arginine.

44. The method of claim 42, wherein the other therapeutic agent is a mito cocktail.
